(19) United States
(12) Patent Application Publication
(10) Pub. No.: US 2025/0204876 A1
(43) Pub. Date: Jun. 26, 2025

(54) INFORMATION PROCESSING APPARATUS, OPERATION METHOD OF INFORMATION PROCESSING APPARATUS, AND PROGRAM

(71) Applicant: FUJIFILM Corporation, Tokyo (JP)

(72) Inventor: Yuta HIASA, Tokyo (JP)

(73) Assignee: FUJIFILM Corporation, Tokyo (JP)

(21) Appl. No.: 18/985,049

(22) Filed: Dec. 18, 2024

(30) Foreign Application Priority Data
Dec. 22, 2023 (JP) 2023-217180

(51) Int. Cl. A61B 6/46; A61B 6/00; G06T 7/00; G06T 7/11; G06T 7/187; G06T 11/00; G06T 11/60; G06V 10/764; G06V 20/70

(52) U.S. Cl. CPC A61B 6/463; A61B 6/5294; G06T 7/0012; G06T 7/11; G06T 7/187; G06T 11/001; G06T 11/60; G06V 10/764; G06V 20/70; G06T 2200/24; G06T 2207/10024; G06T 2207/10081; G06T 2207/20104; G06T 2207/30064; G06T 2207/30096; G06T 2210/41; G06T 2210/62; G06V 2201/032

(57) ABSTRACT

An information processing apparatus uses a multi-disease detection model for detecting a plurality of disease regions from a medical image to acquire, for each disease, a degree of certainty representing at least any of presence or absence of a disease or a degree of the disease for each pixel of the medical image, creates degree-of-certainty distribution information for visualizing a distribution of the degree of certainty for each disease, acquires a threshold value to be applied to the degree of certainty, and superimposes and displays at least any of a first color or a first opacity on the medical image for a pixel of a first image having the degree of certainty of the same value as the threshold value, for any disease.

### CROSS-REFERENCE TO RELATED APPLICATION

The present application claims priority under 35 U.S.C § 119(a) to Japanese Patent Application No. 2023-217180 filed on Dec. 22, 2023, which is hereby expressly incorporated by reference, in its entirety, into the present application.

### BACKGROUND OF THE INVENTION

### 1. Field of the Invention

The present invention relates to an information processing apparatus, an operation method of an information processing apparatus, and a program.

### 2. Description of the Related Art

In recent years, diagnosis support for medical images using AI technology has been put into practical use. For example, a chest disease, such as a nodule, a pneumothorax, and a pleural effusion, is detected from a plain chest X-ray image, and, for a disease region detected as a disease from the plain chest X-ray image, information using a heat map representing a distribution of a degree of certainty and the like and a rectangle surrounding the disease region is generated, and superimposed and displayed on a medical image. Note that AI is an abbreviation for artificial intelligence.

JP2021-029387A discloses a medical information processing apparatus that determines a priority of a detection region of a disease based on a predetermined condition and changes a display form of the detection region according to the priority, in response to a problem in that, in a case in which multiple types of detection regions are displayed at once, the visibility of each detection region is lowered.

### SUMMARY OF THE INVENTION

Heat map output of a disease detection model for a medical image is understood as a distribution of a degree of certainty representing the presence or absence of a target disease for each pixel of an input image. In general, in a case in which the target disease is a single disease, a heat map to which a pseudo color is applied according to a value of the degree of certainty is superimposed and displayed on the medical image. As a result, the suspiciousness of the disease is clearly presented to an interpreter.

However, in a case in which the heat map output of the disease detection model is extended to a multi-disease detection model in which a plurality of diseases are detected, the following problems are present. In a case in which the heat map is displayed for each disease, the number of heat maps to be interpreted increases according to the number of target diseases, and there is a concern about a decrease in efficiency of interpretation.

In a case in which a plurality of diseases are integrated and displayed as one heat map, there is a concern about a decrease in readability of each disease in a case in which a plurality of diseases exist at the same position and in a case in which a plurality of diseases exist at adjacent positions.

The medical information processing apparatus disclosed in JP2021-029387A is highly dependent on the performance of the priority determination of the disease, and there is a possibility that an important disease is given a relatively lower priority. In addition, the medical information processing apparatus disclosed in JP2021-029387A does not take into consideration a problem in that, in a two-dimensional medical image, the visibility of each disease is lowered in a case in which diseases overlap in a depth direction.

The present invention has been made in view of such circumstances, and an object of the present invention is to provide an information processing apparatus, an operation method of an information processing apparatus, and a program with which visualization of each detected disease is achieved in display of a plurality of disease regions detected from one medical image.

According to a first aspect of the present disclosure, there is provided an information processing apparatus comprising: one or more processors; and one or more memories that store instructions to be executed by the one or more processors, in which the one or more processors acquire a medical image, use a multi-disease detection model for detecting a plurality of disease regions from the medical image to acquire, for each disease, a degree of certainty representing at least any of presence or absence of a disease or a degree of the disease for each pixel of the medical image, create degree-of-certainty distribution information for visualizing a distribution of the degree of certainty for each detected disease, acquire a threshold value to be applied to the degree of certainty, and perform control of superimposing at least any of a first color or a first opacity on the medical image for a pixel of a first image having the degree of certainty of the same value as the threshold value and displaying the superimposed image on a display device, for any disease.

With the information processing apparatus according to the first aspect of the present disclosure, the pixel of the first image on which at least any of the first color or the first opacity is superimposed functions as a contour of the disease region. As a result, in the display of the plurality of disease regions detected from one medical image, the visualization of each detected disease is achieved.

The disease region may include a state different from a normal state region. Examples of the disease region include a region including a color, a texture, unevenness, and the like.

An example of the degree-of-certainty distribution information is a heat map in which different colors are used to represent differences in degree of certainty. The difference in color may be a difference in hue or a difference in brightness.

A second aspect of the present disclosure provides the information processing apparatus according to the first aspect, in which the one or more processors may acquire a threshold value range having a pre-specified upper limit value exceeding the threshold value and a pre-specified lower limit value less than the threshold value, and classify a pixel having the degree of certainty within the threshold value range as the pixel of the first image.

According to such an aspect, the number of the pixels of the first image that functions as the contour of the disease region is relatively increased. As a result, the contour of the disease region is emphasized.

A third aspect of the present disclosure provides the information processing apparatus according to the first aspect, in which the one or more processors may classify a pixel located at a distance within a specified range from a pixel classified as the pixel of the first image, as the pixel of the first image.

According to such an aspect, the number of the pixels of the first image that functions as the contour of the disease region is relatively increased. As a result, the contour of the disease region is emphasized.

A fourth aspect of the present disclosure provides the information processing apparatus according to any one of the first to third aspects, in which the one or more processors may perform control of, for a pixel of a second image of which the degree of certainty exceeds the degree of certainty in the pixel of the first image, superimposing and displaying at least any of a second color corresponding to a maximum value of the degree of certainty or a second opacity corresponding to the maximum value of the degree of certainty on the medical image, for any disease.

According to such an aspect, the pixel of the second image is distinguished from the pixel of the first image in at least any of a color or an opacity. As a result, the disease region for each disease is emphasized.

A fifth aspect of the present disclosure provides the information processing apparatus according to the fourth aspect, in which the one or more processors may set a third opacity lower than the first opacity for the pixel of the second image in a case in which a pixel adjacent to the pixel of the second image is classified as the pixel of the first image.

According to such an aspect, transmittance in the contour of the disease region is relatively reduced. As a result, the contour of the disease region is emphasized.

A sixth aspect of the present disclosure provides the information processing apparatus according to any one of the first to fifth aspects, in which the one or more processors may perform control of causing the medical image to be displayed with a non-superimposed color and a non-superimposed opacity for a pixel of a third image of which the degree of certainty is less than the degree of certainty in the pixel of the first image, for any disease.

According to such an aspect, visual recognition of the medical image in a non-disease region is not hindered.

A seventh aspect of the present disclosure provides the information processing apparatus according to any one of the first to sixth aspects, in which the one or more processors may acquire a maximum value of the degree of certainty of each disease, determine whether or not the maximum value of the degree of certainty exceeds the threshold value for each disease, and perform control of displaying text representing a disease name and the maximum value of the degree of certainty in a case in which the maximum value of the degree of certainty exceeds the threshold value.

According to such an aspect, a user can grasp the maximum value of the degree of certainty of each disease.

An eighth aspect of the present disclosure provides the information processing apparatus according to any one of the first to seventh aspects, in which the one or more processors may execute labeling processing on one or more connected regions including a plurality of consecutive pixels of which a value of the degree of certainty of each disease exceeds the threshold value, determine whether or not a maximum value of the degree of certainty exceeds the threshold value for each connected region of each disease, receive an input of a user who designates the connected region, and perform control of displaying the maximum value of the degree of certainty of each disease in the designated connected region.

According to such an aspect, the user can grasp the maximum value of the degree of certainty of each disease, for each connected region designated by the user.

A ninth aspect of the present disclosure provides the information processing apparatus according to any one of the first to eighth aspects, in which the one or more processors may receive an input of a user who designates a pixel in the medical image, and perform control of, in a case in which the degree of certainty of each disease in the designated pixel exceeds the threshold value, displaying a value of the degree of certainty.

According to such an aspect, the user can grasp the maximum value of the degree of certainty of each disease, for each pixel designated by the input from the user.

A tenth aspect of the present disclosure provides the information processing apparatus according to any one of the first to ninth aspects, in which the one or more processors may execute labeling processing on one or more connected regions including a plurality of consecutive pixels of which a value of the degree of certainty of each disease exceeds the threshold value, and perform control of displaying at least any of a disease name or the degree of certainty of the connected region for each connected region of each disease.

According to such an aspect, the user can grasp the disease name and the degree of certainty of each disease, for each connected region.

An eleventh aspect of the present disclosure provides the information processing apparatus according to the tenth aspect, in which the one or more processors may perform control of displaying at least any of the disease name for each connected region or the degree of certainty of the connected region outside a detection target region in the medical image.

According to such an aspect, text information does not hinder the visibility of the medical image.

A twelfth aspect of the present disclosure provides the information processing apparatus according to any one of the first to eleventh aspects, in which the one or more processors may execute labeling processing on one or more connected regions including a plurality of consecutive pixels of which the degree of certainty of each disease exceeds the threshold value, and perform control of displaying all the degrees of certainty of each disease for each connected region.

According to such an aspect, the user can grasp all the degrees of certainty of each disease, for each connected region.

A thirteenth aspect of the present disclosure provides the information processing apparatus according to any one of the first to twelfth aspects, in which the one or more processors may execute labeling processing on one or more connected regions including a plurality of consecutive pixels of which the degree of certainty of each disease exceeds the threshold value, and perform control of displaying text information obtained by integrating a plurality of disease names in an overlapping portion of each disease in a case in which the overlapping portion exceeds a specified value, for each connected region of each disease.

According to such an aspect, the visibility of the text information can be relatively improved for a plurality of disease regions overlapping each other. In addition, the text information does not hinder the visibility of the medical image.

A fourteenth aspect of the present disclosure provides the information processing apparatus according to any one of the first to thirteenth aspects, in which the one or more processors may replace a value of a degree of certainty of a pixel constituting the degree-of-certainty distribution information with a maximum value of a degree of certainty in a local region located within a specified range from the pre-specified pixel.

According to such an aspect, pixels in a foreground region of the medical image on which the degree-of-certainty distribution information is superimposed are not isolated, and the visibility of the degree-of-certainty distribution information can be improved.

A fifteenth aspect of the present disclosure provides the information processing apparatus according to any one of the first to fourteenth aspects, in which the one or more processors may receive an input of a user for making a change to control of displaying the degree-of-certainty distribution information.

According to such an aspect, selective switching between the visualization display of the disease region and the display of the degree-of-certainty distribution information is implemented.

According to a sixteenth aspect of the present disclosure, there is provided an operation method of an information processing apparatus, the method being executed by a computer functioning as the information processing apparatus and comprising: a step of acquiring a medical image; a step of using a multi-disease detection model for detecting a plurality of disease regions from the medical image to acquire, for each disease, a degree of certainty representing at least any of presence or absence of a disease or a degree of the disease for each pixel of the medical image; a step of creating degree-of-certainty distribution information for visualizing a distribution of the degree of certainty for each detected disease; a step of acquiring a threshold value to be applied to the degree of certainty; and a step of performing control of superimposing at least any of a first color or a first opacity on the medical image for a pixel of a first image having the degree of certainty of the same value as the threshold value and displaying the superimposed image on a display device, for any disease.

With the operation method of the information processing apparatus according to the sixteenth aspect of the present disclosure, it is possible to obtain the same effects as the information processing apparatus according to the first aspect of the present disclosure.

In the operation method of the information processing apparatus according to the sixteenth aspect, the same items as those specified in the second to fifteenth aspects can be combined as appropriate. In this case, the component responsible for the specified processing or function in the information processing apparatus can be understood as a component of the operation method of the information processing apparatus responsible for the corresponding processing or function.

According to a seventeenth aspect of the present disclosure, there is provided a program causing a computer functioning as an information processing apparatus to implement: a function of acquiring a medical image; a function of using a multi-disease detection model for detecting a plurality of disease regions from the medical image to acquire, for each disease, a degree of certainty representing at least any of presence or absence of a disease or a degree of the disease for each pixel of the medical image; a function of creating degree-of-certainty distribution information for visualizing a distribution of the degree of certainty for each detected disease; a function of acquiring a threshold value to be applied to the degree of certainty; and a function of performing control of superimposing at least any of a first color or a first opacity on the medical image for a pixel of a first image having the degree of certainty of the same value as the threshold value and displaying the superimposed image on a display device, for any disease.

With the program according to the seventeenth aspect of the present disclosure, it is possible to obtain the same effects as the information processing apparatus according to the first aspect of the present disclosure.

In the program according to the seventeenth aspect, the same items as those specified in the second to fifteenth aspects can be combined as appropriate. In this case, the component responsible for the specified processing or function in the information processing apparatus can be understood as a component of the program responsible for the corresponding processing or function.

According to an eighteenth aspect of the present disclosure, there is provided an information processing apparatus comprising: one or more processors; and one or more memories that store instructions to be executed by the one or more processors, in which the one or more processors acquire a medical image, use a multi-disease detection model for detecting a plurality of disease regions from the medical image to acquire, for each disease, a degree of certainty representing at least any of presence or absence of a disease or a degree of the disease for each pixel of the medical image, acquire a threshold value to be applied to the degree of certainty, acquire information on a region for each anatomical structure from the medical image, acquire a maximum value of the degree of certainty of each disease, for the region for each anatomical structure, and store a combination of an anatomical structure name, a disease name, and the maximum value of the degree of certainty in a case in which the maximum value of the degree of certainty of each disease in the region for each anatomical structure exceeds the threshold value.

With the image processing apparatus according to the eighteenth aspect of the present disclosure, support for creating a finding report is implemented.

According to the present invention, the pixel of the first image on which at least any of the first color or the first opacity is superimposed functions as the contour of the disease region. As a result, in the display of the plurality of disease regions detected from one medical image, the visualization of each detected disease is achieved.

### BRIEF DESCRIPTION OF THE DRAWINGS

FIG. 1 is an overall configuration diagram of a medical information processing system.

FIG. 2 is a functional block diagram showing an electric configuration of a medical information processing apparatus according to a first embodiment.

FIG. 3 is a block diagram showing an example of a hardware configuration of the medical information processing apparatus illustrated in FIG. 2.

FIG. 4 is a flowchart showing a procedure of a medical information processing method according to the first embodiment.

FIG. 5 is a schematic diagram of a heat map image and an all-findings integrated image according to the first embodiment.

FIG. 6 is a schematic diagram of a medical image on which first superimposition information is superimposed.

FIG. 7 is a schematic diagram of a medical image on which second superimposition information and third superimposition information are superimposed.

FIG. 8 is a functional block diagram showing an electric configuration of a medical information processing apparatus according to a second embodiment.

FIG. 9 is a schematic diagram of a medical image on which text information representing a maximum value of a degree of certainty of each disease is superimposed.

FIG. 10 is a functional block diagram showing an electric configuration of a medical information processing apparatus according to a third embodiment.

FIG. 11 is a schematic diagram of a medical image on which a maximum value of a degree of certainty of each disease in a designated region is superimposed.

FIG. 12 is a functional block diagram showing an electric configuration of a medical information processing apparatus according to a fourth embodiment.

FIG. 13 is a functional block diagram showing an electric configuration of a medical information processing apparatus according to a fifth embodiment.

FIG. 14 is a schematic diagram of a medical image on which a disease name and a degree of certainty of each disease are superimposed for each region.

FIG. 15 is a schematic diagram showing a modification example of the text information illustrated in FIG. 14.

FIG. 16 is a schematic diagram showing another modification example of the text information illustrated in FIG. 14.

FIG. 17 is a functional block diagram showing an electric configuration of a medical information processing apparatus according to a sixth embodiment.

FIG. 18 is a schematic diagram of integrated text information.

FIG. 19 is a functional block diagram showing an electric configuration of a medical information processing apparatus according to a seventh embodiment.

### DESCRIPTION OF THE PREFERRED EMBODIMENTS

Hereinafter, preferred embodiments of the present invention will be described with reference to the accompanying drawings. In the present specification, the same components are denoted by the same reference numerals, and duplicate description thereof will be omitted as appropriate. In addition, in the following embodiments, in a case in which a plurality of components are described and listed, it can be interpreted that at least one of the plurality of components is included.

### Configuration Example of Medical Information Processing System

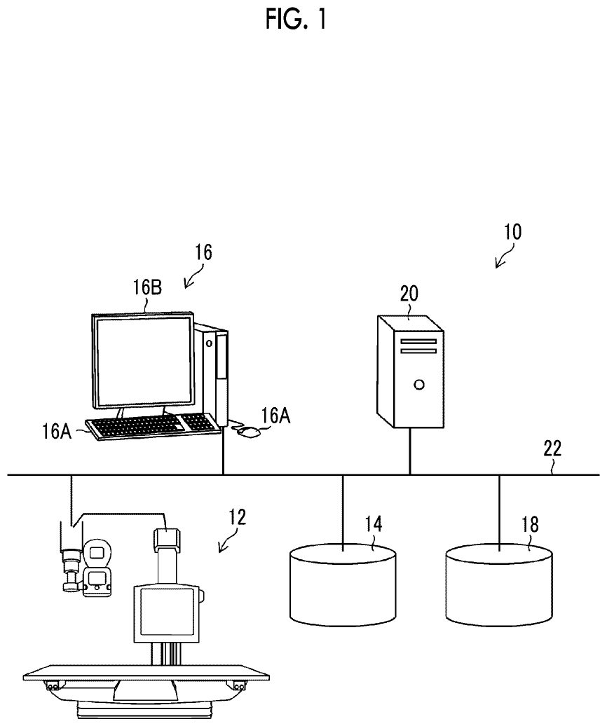

FIG. 1 is an overall configuration diagram of a medical information processing system. A medical information processing system 10 illustrated in FIG. 1 comprises a medical image capturing apparatus 12, a medical image database 14, a user terminal device 16, an interpretation report database 18, and a medical information processing apparatus 20.

The medical image capturing apparatus 12, the medical image database 14, the user terminal device 16, the interpretation report database 18, and the medical information processing apparatus 20 are electrically connected to each other via a network 22 so as to be able to freely transmit and receive to and from each other. A communication form of the network 22 may be wired or wireless.

An example of the network 22 is a LAN that connects various devices in a medical institution. The network 22 may be a WAN that connects LANs of a plurality of medical institutions. Note that LAN is an abbreviation for local area network. WAN is an abbreviation for wide area network.

The medical image capturing apparatus 12 images an examination target part of a subject and generates a medical image. FIG. 1 illustrates an X-ray imaging apparatus as the medical image capturing apparatus 12. Other examples of the medical image capturing apparatus 12 include a CT apparatus, an MRI apparatus, a PET apparatus, an ultrasound apparatus, a CR apparatus using a flat panel X-ray detector, and an endoscope apparatus. Note that CT is an abbreviation for computed tomography. MRI is an abbreviation for magnetic resonance imaging. PET is an abbreviation for positron emission tomography. CR is an abbreviation for computed radiography.

The medical image database 14 is a database for managing medical images generated by being captured using the medical image capturing apparatus 12. The medical image database 14 comprises a large-capacity storage device in which the medical images are stored. The medical image database 14 comprises a computer in which software that provides a function of a database management system is incorporated. Note that the term “software” is synonymous with the term “program”.

The medical image may be a two-dimensional image generated using an X-ray imaging apparatus or the like, or may be a three-dimensional reconstructed image generated using a CT apparatus or the like.

As a format of the medical image, a DICOM standard may be applied. The medical image may have accessory information such as DICOM tag information defined in the DICOM standard. Note that DICOM is an abbreviation for digital imaging and communications in medicine. Here, the term “image” in the present specification may include the meaning of image data, which is a signal representing an image, as well as the meaning of an image itself, such as a photograph.

The user terminal device 16 is a terminal device that allows a user, such as a doctor, to create an interpretation report and view the interpretation report. Viewer software used in a case in which the user browses a medical image is installed in the user terminal device 16. A computer is applied as the user terminal device 16. The computer applied as the user terminal device 16 may be a workstation or a tablet terminal.

An input device 16A and a display device 16B are connected to the user terminal device 16. In FIG. 1, a keyboard and a mouse are illustrated as the input device 16A. The display device 16B may comprise a touch panel type display, and the input device 16A and the display device 16B may be integrated together.

A liquid crystal display, an organic EL display, a projector, or the like can be applied as the display device 16B. Any combination of a plurality of devices can be applied as the display device 16B. Note that the term “EL” of the organic EL display is an abbreviation for electro-luminescence.

A doctor who is a user can issue an instruction to display a medical image by using the input device 16A. The user terminal device 16 receives an instruction from the user and displays the medical images on the display device 16B in accordance with the instruction from the user. The doctor creates a key image that is determined to be important in the interpretation based on the content of findings from among the medical images displayed on the display device 16B. Further, the doctor uses the input device 16A to input findings representing an interpretation result of the medical image. In this way, the doctor uses the user terminal device 16 to create an interpretation report including the key image and the findings.

The interpretation report database 18 is a database for managing the interpretation report created by the doctor. The interpretation report database 18 comprises a large-capacity storage device in which the interpretation report is stored. The interpretation report database 18 comprises a computer in which software that provides a function of a database management system is incorporated. The medical image database 14 and the interpretation report database 18 may be configured by using one computer.

The medical information processing apparatus 20 functions as a medical image processing apparatus that executes various types of processing on a medical image. For example, the medical information processing apparatus 20 generates various types of information to be superimposed on the medical image, and displays the medical image on which the various types of information are superimposed on a display device. The display device that displays the medical image may be a display device 16B provided in the user terminal device 16.

As the medical information processing apparatus 20, a computer comprising one or more processors and one or more memories that store a program including one or more instructions to be executed by the one or more processors is applied. The computer applied as the medical information processing apparatus 20 may be a workstation or the like. The medical information processing apparatus 20 may be a virtual machine.

### Configuration Example of Medical Information Processing Apparatus According to First Embodiment

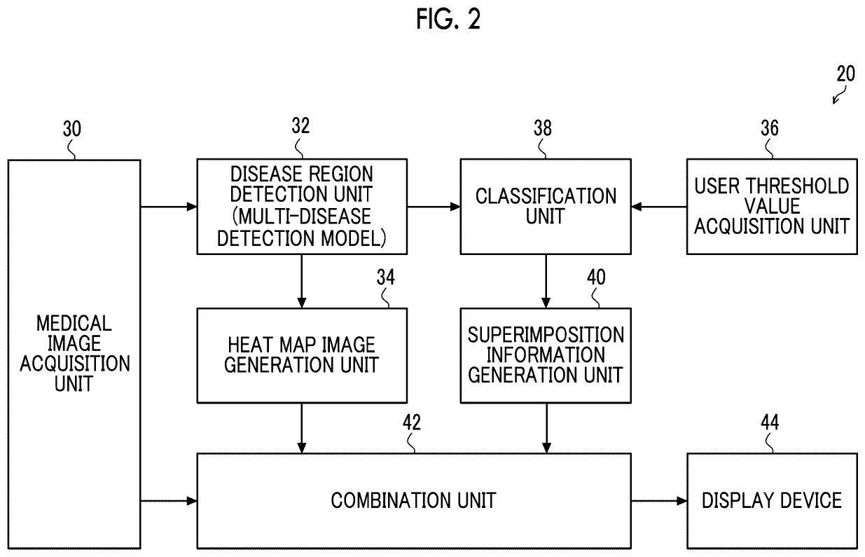

FIG. 2 is a functional block diagram showing an electric configuration of a medical information processing apparatus according to a first embodiment. The medical information processing apparatus 20 has a function of visualizing heat map output of a multi-disease detection model for detecting a plurality of disease regions from the medical image.

The medical information processing apparatus 20 comprises a medical image acquisition unit 30, a disease region detection unit 32, a heat map image generation unit 34, a user threshold value acquisition unit 36, a classification unit 38, a superimposition information generation unit 40, and a combination unit 42.

The medical image acquisition unit 30 acquires a medical image to be processed. The medical image acquisition unit 30 may acquire a medical image from the medical image database 14 or may acquire a medical image from the medical image capturing apparatus 12.

The medical image acquired by the medical image acquisition unit 30 may be a reconstructed image or raw data. In the present embodiment, an example is shown in which the medical image acquisition unit 30 acquires a two-dimensional X-ray image as the medical image. Pixels of the two-dimensional medical image are specified using coordinate values of a two-dimensional coordinate system applied to the medical image. An example of the two-dimensional coordinate system is a two-dimensional orthogonal coordinate system. The origin of the two-dimensional orthogonal coordinate system is appropriately defined. The coordinate values of the two-dimensional coordinate system can be understood as positions of the respective pixels.

The medical image acquisition unit 30 may have a function of a processing unit that executes processing on the acquired medical image. For example, the medical image acquisition unit 30 may acquire raw data as the medical image and generate a reconstructed image from the raw data.

The disease region detection unit 32 detects a disease region from the medical image acquired by using the medical image acquisition unit 30, and acquires a degree of certainty representing at least any of the presence or absence of a disease or a degree of a disease for each pixel constituting the medical image. The disease region detection unit 32 acquires the degree of certainty for each disease.

For example, in a case in which the degree of certainty is represented as a value of 0 or more and 100 or less, a value of the degree of certainty of 0 means that there is no disease, and a value of the degree of certainty of 100 means that there is a disease. In a case in which the degree of certainty is a value greater than 0 and less than 100, a value of the degree of certainty that is relatively large represents that the probability of the disease is relatively high, and a value of the degree of certainty that is relatively small represents that the probability of the disease is relatively low. The degree of certainty may represent a degree of a disease, such as severity of the disease.

The disease region detection unit 32 may be a multi-disease detection model for detecting a plurality of different diseases from one medical image. A trained model may be applied as the multi-disease detection model. A deep learning model such as CNN may be applied as the trained model. Note that CNN is an abbreviation for convolutional neural network.

The disease region detection unit 32 may detect a plurality of diseases from one medical image using one multi-disease detection model. The disease region detection unit 32 may detect a plurality of diseases with a plurality of disease detection models, in which the diseases to be detected are different, for each disease.

The heat map image generation unit 34 generates a heat map image representing a distribution of the degree of certainty for each pixel. In a case in which a plurality of diseases are detected in the disease region detection unit 32, a heat map image is generated for each disease. A plurality of the heat map images are integrated for all findings.

Pixels constituting the heat map image are represented using a two-dimensional coordinate system applied to the medical image. That is, the pixels constituting the medical image are stored in association with the degree of certainty of each disease and the pixels constituting the heat map image. The heat map image described in the embodiment is an example of degree-of-certainty distribution information representing a distribution of the degree of certainty.

The user threshold value acquisition unit 36 acquires a user threshold value used in classifying the degree of certainty of each disease acquired by using the disease region detection unit 32. The user threshold value acquisition unit 36 may acquire the user threshold value input by the user, or may read out and acquire the user threshold value stored in advance for each specified condition, according to the condition.

The classification unit 38 classifies the degree of certainty of each disease for each pixel of the medical image by using the user threshold value. The classification unit 38 classifies a pixel of which the degree of certainty matches the user threshold value into a first class. The classification unit 38 classifies a pixel of which the degree of certainty exceeds the user threshold value into a second class. The classification unit 38 classifies a pixel of which the degree of certainty is less than the user threshold value into a third class.

That is, the classification unit 38 classifies a pixel having a degree of certainty exceeding the degree of certainty in the pixel classified into the first class, into the second class. The classification unit 38 classifies a pixel having a degree of certainty less than the degree of certainty in the pixel classified into the first class, into the third class.

The classification unit 38 classifies a pixel of which the degree of certainty is within a user threshold value range into the first class, classifies a pixel of which the degree of certainty exceeds an upper limit value of the user threshold value range into the second class, and classifies a pixel of which the degree of certainty is less than a lower limit value of the user threshold value range into the third class. The classification unit 38 stores the classification result for each pixel.

For example, in a case in which the degree of certainty is represented by a value of 0 or more and 100 or less and the user threshold value is 50, a pixel of which the degree of certainty is 50 is classified into the first class, a pixel of which the degree of certainty exceeds 50 is classified into the second class, and a pixel of which the degree of certainty is less than 50 is classified into the third class. The pixel classified into the first class described in the embodiment is an example of a pixel of a first image. The pixel classified into the second class described in the embodiment is an example of a pixel of a second image. The pixel classified into the third class described in the embodiment is an example of a pixel of a third image.

The user threshold value acquisition unit 36 may acquire a user threshold value range that includes, in the user threshold value, a value in the vicinity of the user threshold value. That is, the user threshold value acquisition unit 36 may acquire an upper limit value of the user threshold value range and a lower limit value of the user threshold value range.

The classification unit 38 may use the user threshold value range to classify each pixel constituting the medical image based on the degree of certainty of each disease for each pixel. For example, in a case in which a range of plus or minus 1.0% of the user threshold value is defined as the vicinity of the user threshold value, a range of 49.5 or more and 50.5 or less is acquired as the user threshold value range.

The classification unit 38 may classify a pixel of which the degree of certainty is 49.5 or more and 50.5 or less into the first class. The classification unit 38 may classify a pixel of which the degree of certainty exceeds 50.5 into the second class and classify a pixel of which the degree of certainty is less than 49.5 into the second class.

The classification unit 38 may classify a pixel of which a distance from the pixel classified into the first class is within a specified range into the first class. For example, the classification unit 38 may classify all pixels within 1.0 millimeter from the pixel classified into the first class into the first class regardless of the degree of certainty.

The superimposition information generation unit 40 generates superimposition information corresponding to the class for each pixel. The superimposition information generation unit 40 generates first superimposition information in which a fixed first color and a value of a first opacity are superimposed on the input medical image for the pixels classified into the first class. The fixed first color is preferably a color that can be visually distinguished from a color used in the heat map image.

The superimposition information generation unit 40 generates second superimposition information in which a second color and a value of a second opacity are superimposed on the input medical image according to a maximum value of the degree of certainty among the diseases classified into the second class, for the pixels classified into the second class.

The superimposition information generation unit 40 generates third superimposition information in which the input medical image is displayed as it is for the pixels classified into the third class. For example, the superimposition information generation unit 40 may execute processing of not generating superimposition information to be superimposed on the input medical image for the pixels classified into the third class. The third superimposition information in which the input medical image described in the embodiment is displayed as it is is an example of processing of non-superimposing the color and the opacity executed for the pixels of the third class.

The combination unit 42 generates a composite image in which the heat map image is superimposed on the input medical image and further, at least any of the first superimposition information, the second superimposition information, or the third superimposition information is superimposed on the heat map image. For example, the combination unit 42 may generate a composite image in which the first superimposition information is superimposed on the heat map image. The combination unit 42 may generate a composite image in which the first superimposition information and the second superimposition information are superimposed on the input medical image. The combination unit 42 displays the composite image on a display device 44.

The medical information processing apparatus 20 may comprise a display controller that generates a display signal representing the composite image generated by the combination unit 42. The display device 44 may acquire a signal representing the composite image and convert the signal into a display signal representing the composite image.

The display device 44 illustrated in FIG. 2 may be a display device connected to a computer connected via the network 22 illustrated in FIG. 1. For example, the display device 44 illustrated in FIG. 2 may be the display device 16B connected to the user terminal device 16 illustrated in FIG. 1.

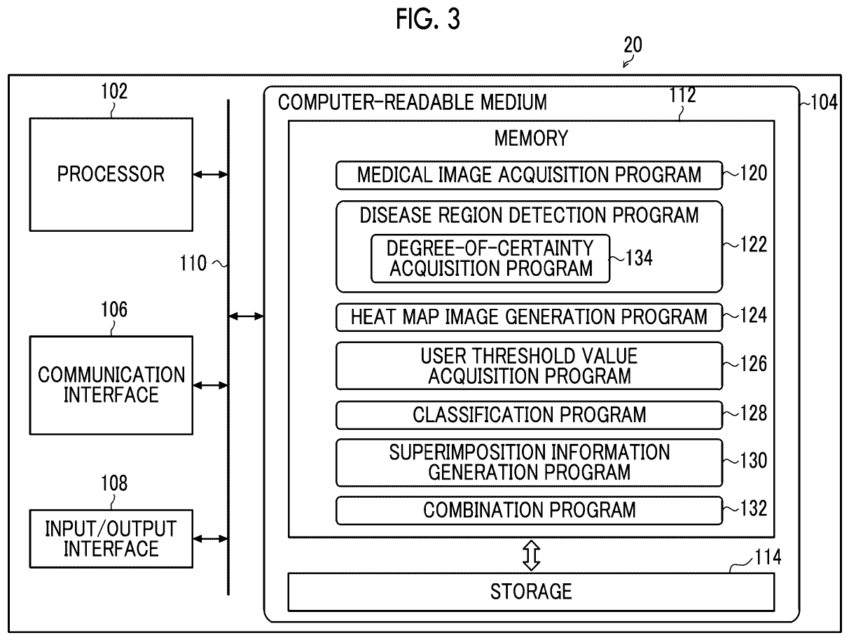

FIG. 3 is a block diagram showing an example of a hardware configuration of the medical information processing apparatus illustrated in FIG. 2. The medical information processing apparatus 20 comprises a processor 102, a computer-readable medium 104, a communication interface 106, an input/output interface 108, and a bus 110.

The processor 102 includes a CPU. The processor 102 may include a GPU. Note that CPU is an abbreviation for central processing unit. GPU is an abbreviation for graphics processing unit.

The processor 102 is connected to the computer-readable medium 104, the communication interface 106, and the input/output interface 108 via the bus 110.

In the medical information processing apparatus 20, the processor 102 executes a program stored in the computer-readable medium 104 to implement various functions. Note that the term “program” is synonymous with the term “software”.

The computer-readable medium 104 comprises a memory 112 which is a main memory and a storage 114 which is an auxiliary memory. A semiconductor memory, a hard disk apparatus, a solid state drive apparatus, and the like can be applied to the computer-readable medium 104. Any combination of a plurality of apparatuses can be applied to the computer-readable medium 104.

Note that the hard disk apparatus can be referred to as an HDD which is an abbreviation for hard disk drive in English. The solid state drive apparatus can be referred to as an SSD which is an abbreviation for solid state drive in English.

The medical information processing apparatus 20 executes data communication with an external device via the communication interface 106. Various standards such as USB can be applied to the communication interface 106. Either wired communication or wireless communication may be applied to a communication form of the communication interface 106. Note that USB is an abbreviation for universal serial bus and is a registered trademark.

The memory 112 of the computer-readable medium 104 stores a medical image acquisition program 120, a disease region detection program 122, a heat map image generation program 124, a user threshold value acquisition program 126, a classification program 128, a superimposition information generation program 130, and a combination program 132, which are executed by the processor 102. The disease region detection program 122 may include a degree-of-certainty acquisition program 134 that calculates a degree of certainty of each disease for each pixel.

The medical image acquisition program 120 is applied to the medical image acquisition unit 30 illustrated in FIG. 2 and implements a function of acquiring a medical image. The disease region detection program 122 and the degree-of-certainty acquisition program 134 implement a function of detecting a disease region from a medical image and acquiring a degree of certainty of each disease for each pixel as a detection result. The disease region detection program 122 and the degree-of-certainty acquisition program 134 are trained multi-disease detection models applied to the disease region detection unit 32.

The heat map image generation program 124 is applied to the heat map image generation unit 34 and implements a function of generating a heat map. The user threshold value acquisition program 126 is applied to the user threshold value acquisition unit 36 and implements a function of acquiring a user threshold value. The classification program 128 is applied to the classification unit 38 and implements a function of classifying pixels based on a degree of certainty of each disease for each pixel.

The superimposition information generation program 130 is applied to the superimposition information generation unit 40, and the combination program 132 that implements a function of generating superimposition information is applied to the combination unit 42 and implements a function of generating a composite image in which a heat map image is superimposed on the input medical image and further, superimposition information for each class is superimposed on the heat map image.

Various programs stored in the computer-readable medium 104 include one or more instructions. The computer-readable medium 104 stores various data, various parameters, and the like used in a case in which various types of programs are executed.

Here, examples of a hardware structure of the processor 102 include a CPU, a GPU, a programmable logic device (PLD), and an application specific integrated circuit (ASIC). The CPU is a general-purpose processor that executes a program and acts as various functional units. The GPU is a processor specialized in image processing.

The PLD is a processor capable of changing a configuration of an electric circuit after manufacturing a device. An example of the PLD is a field programmable gate array (FPGA). The ASIC is a processor comprising a dedicated electric circuit specifically designed to execute a specific process.

One processing unit may be configured by one of these various processors or may be composed of two or more processors of the same type or different types. Examples of a combination of various processors include a combination of one or more FPGAs and one or more CPUs, and a combination of one or more FPGAs and one or more GPUs. Another example of a combination of various processors includes a combination of one or more CPUs and one or more GPUs.

A plurality of functional units may be configured by using one processor. As an example of configuring a plurality of functional units by using one processor, there is an aspect in which, as typified by a computer such as a client or a server, a combination of one or more CPUs and software such as a system on a chip (SoC) is applied to configured one processor, and the processor is caused to act as a plurality of functional units.

As another example of configuring a plurality of functional units by using one processor, there is an aspect in which a processor that realizes functions of an entire system including a plurality of functional units by using one IC chip is used. Note that IC is an abbreviation for integrated circuit.

As described above, the various processing units are configured using one or more of the various processors as a hardware structure. Furthermore, the hardware structure of the above described various processors is, more specifically, an electric circuit (circuitry) in which circuit elements such as semiconductor elements are combined.

### Procedure of Medical Information Processing Method According to First Embodiment

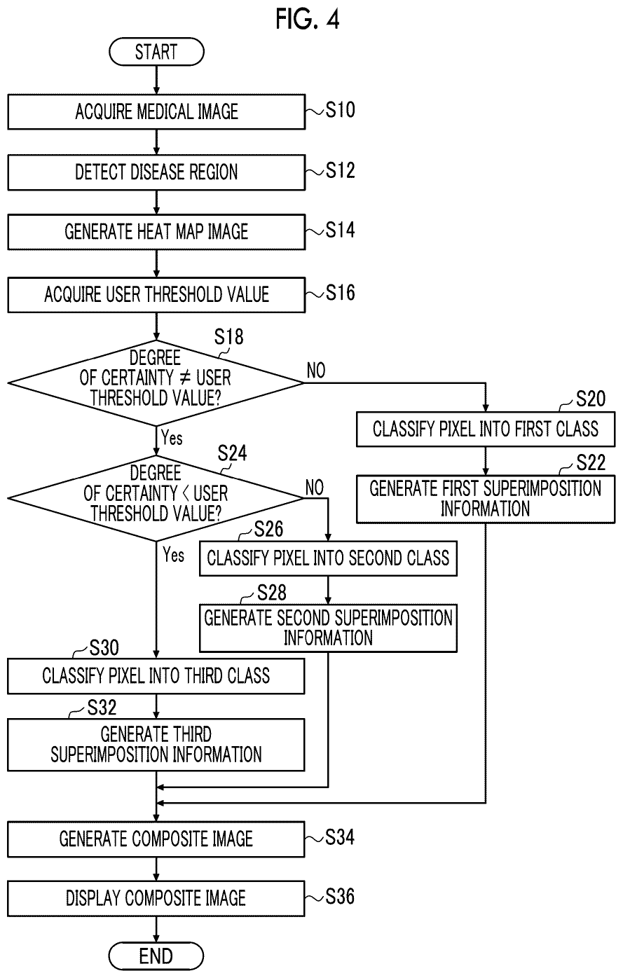

FIG. 4 is a flowchart showing a procedure of a medical information processing method according to the first embodiment. In a medical image acquisition step S10, the medical image acquisition unit 30 illustrated in FIG. 2 acquires a medical image. After the medical image acquisition step S10, the process proceeds to a disease region detection step S12.

In the disease region detection step S12, the disease region detection unit 32 detects one or more disease regions from the acquired medical image. In the disease region detection step S12, a degree of certainty of each disease is acquired for each pixel as a detection result of the disease region. After the disease region detection step S12, the process proceeds to a heat map image generation step S14.

In the heat map image generation step S14, the heat map image generation unit 34 generates a heat map image representing a distribution of the degree of certainty in the medical image for each disease. After the heat map image generation step S14, the process proceeds to a user threshold value acquisition step S16.

In the user threshold value acquisition step S16, the user threshold value acquisition unit 36 acquires a user threshold value. In the user threshold value acquisition step S16, the user threshold value acquisition unit 36 may acquire an upper limit value of a user threshold value range and a lower limit value of a user threshold value range. After the user threshold value acquisition step S16, the process proceeds to a first classification step S18.

The user threshold value acquisition step S16 may be executed before the heat map image generation step S14 is executed, or may be executed in parallel with the heat map image generation step S14. That is, the user threshold value need only be acquired before the first classification step S18 is started.

In the first classification step S18, the classification unit 38 determines, for each pixel and for each disease, whether or not the degree of certainty of each disease for each pixel is different from the user threshold value. In the first classification step S18, the classification unit 38 may determine, for each pixel and for each disease, whether or not the degree of certainty of each disease for each pixel is the same value as the user threshold value.

A pixel of which the degree of certainty is the same as the user threshold value is determined as No, and the process proceeds to a first class decision step S20. In a case in which the user threshold value range is applied, a pixel of which the degree of certainty is within the user threshold value range is determined as No, and the process may proceed to the first class decision step S20.

In the first class decision step S20, the classification unit 38 decides the classification of the pixel of which the degree of certainty is the same as the user threshold value as the first class, and stores the classification result for each pixel and for each disease. In a case in which the user threshold value range is applied, the classification of the pixel of which the degree of certainty is within the user threshold value range may be decided as the first class. After the first class decision step S20, the process proceeds to a first superimposition information generation step S22.

In the first superimposition information generation step S22, the superimposition information generation unit 40 generates first superimposition information for the pixel classified into the first class. After the first superimposition information generation step S22, the process proceeds to a composite image generation step S34.

On the other hand, in the first classification step S18, a pixel of which the degree of certainty exceeds the user threshold value and a pixel of which the degree of certainty is less than the user threshold value are determined as Yes, and the process proceeds to a second classification step S24. In a case in which the user threshold value range is applied, a pixel of which the degree of certainty is less than the lower limit value of the user threshold value range and a pixel of which the degree of certainty exceeds the upper limit value of the user threshold value range are determined as Yes, and the process may proceed to the second classification step S24.

In the second classification step S24, the classification unit 38 determines whether or not the degree of certainty is less than the user threshold value. In the second classification step S24, a pixel of which the degree of certainty exceeds the user threshold value is determined as No, and the process proceeds to a second class decision step S26. In a case in which the user threshold value range is applied, a pixel of which the degree of certainty exceeds the upper limit value of the user threshold value range is determined as No, and the process may proceed to the second class decision step S26.

In the second class decision step S26, the classification unit 38 decides the classification of the pixel of which the degree of certainty exceeds the user threshold value as the second class, and stores the classification result for each pixel and for each disease. In a case in which the user threshold value range is applied, the classification of the pixel of which the degree of certainty exceeds the upper limit value of the user threshold value range may be decided as the second class. After the second class decision step S26, the process proceeds to a second superimposition information generation step S28.

In the second superimposition information generation step S28, the superimposition information generation unit 40 generates second superimposition information for the pixel classified into the second class. After the second superimposition information generation step S28, the process proceeds to the composite image generation step S34.

On the other hand, in the second classification step S24, a pixel of which the degree of certainty is less than the user threshold value is determined as Yes, and the process proceeds to a third class decision step S30. In a case in which the user threshold value range is applied, a pixel of which the degree of certainty is less than the lower limit value of the user threshold value range is determined as Yes, and the process may proceed to the third class decision step S30.

In the third class decision step S30, the classification unit 38 decides the classification of the pixel of which the degree of certainty is less than the user threshold value as the third class, and stores the classification result for each pixel and for each disease. In a case in which the user threshold value range is applied, the classification of the pixel of which the degree of certainty is less than the lower limit value of the user threshold value range may be decided as the third class. After the third class decision step S30, the process proceeds to a third superimposition information generation step S32.

In the third superimposition information generation step S32, the superimposition information generation unit 40 generates third superimposition information for the pixel classified into the third class. In a case in which the third superimposition information is not generated and the pixel classified into the third class is displayed as it is in the medical image, the third superimposition information generation step S32 may be omitted. After the third superimposition information generation step S32, the process proceeds to the composite image generation step S34.

In the composite image generation step S34, the combination unit 42 generates a composite image in which the heat map image is superimposed on the input medical image and further, at least any of the first superimposition information, the second superimposition information, or the third superimposition information is superimposed on the heat map image. That is, in the composite image generation step S34, the superimposition of the second superimposition information on the heat map image may be omitted, and the superimposition of the third superimposition information on the heat map image may be omitted. After the composite image generation step S34, the process proceeds to a composite image display step S36.

In the composite image display step S36, the combination unit 42 displays the composite image on the display device 44. In a case in which the composite image is displayed on the display device 44, the procedure of the medical information processing method is ended. The medical information processing method whose procedure is illustrated in FIG. 4 is an example of an operation method of the information processing apparatus. Each step illustrated in FIG. 4 is an example of a step in which various types of processing are executed.

At least a part of the steps in the procedure of the medical information processing method illustrated in FIG. 4 may include processing according to a signal input in response to an operation of an operator. In addition, the processing of the computer functioning as the information processing apparatus may include processing of acquiring information from an external computer, a measurement device, and the like. The same applies to a procedure of a medical information processing method according to another embodiment.

### Specific Example of Heat Map Display

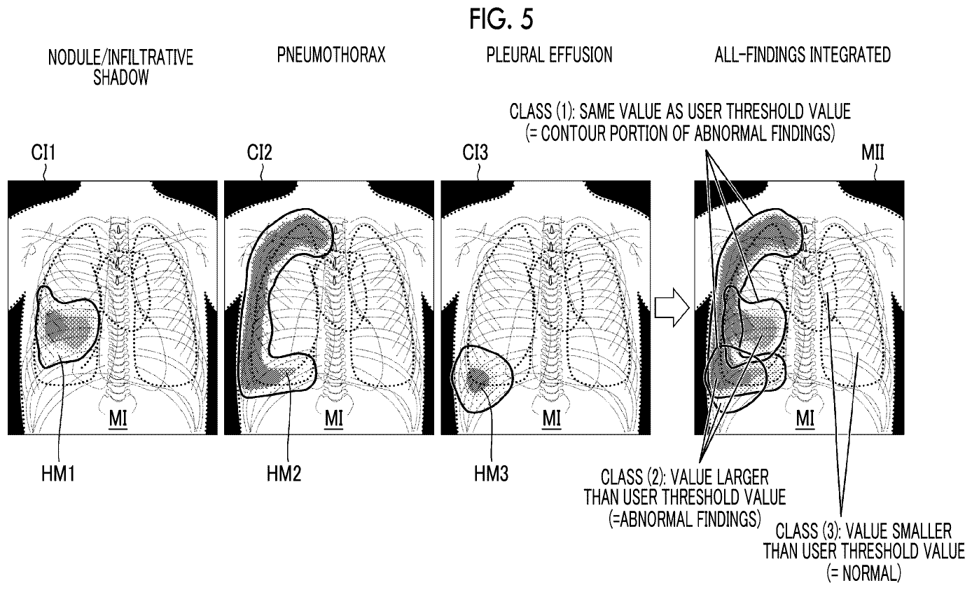

FIG. 5 is a schematic diagram of a heat map image and an all-findings integrated image according to the first embodiment. In FIG. 5, a chest X-ray image is applied as a medical image MI on which the heat map image is superimposed, and a composite image CI1 in which a heat map image HM1 is superimposed on the medical image MI, a composite image CI2 in which a heat map image HM2 is superimposed on the medical image MI, and a composite image CI3 in which a heat map image HM3 is superimposed on the medical image MI are illustrated. In addition, FIG. 5 illustrates an all-findings integrated image MII in which the composite image CI1, the composite image CI2, and the composite image CI3 are integrated. That is, the all-findings integrated image MII illustrated in FIG. 5 is an example of a composite image that is generated in the composite image generation step S34 illustrated in FIG. 4 and that is displayed in the composite image display step S36.

The heat map image HM1 represents a distribution of a degree of certainty of a disease in a case in which a nodule or an infiltrative shadow is detected as the disease. The heat map image HM2 represents a distribution of a degree of certainty of a disease in a case in which a pneumothorax is detected as the disease. The heat map image HM3 represents a distribution of a degree of certainty of a disease in a case in which a pleural effusion is detected as the disease.

In the heat map image HM1, the magnitude of the degree of certainty is represented by using a plurality of colors with different hues, such as red, orange, yellow, green, blue, indigo, and purple. For example, in the heat map image HM1, a color on a relatively red side is used for a pixel of which the degree of certainty is relatively high, and a color on a relatively purple side is used for a pixel of which the degree of certainty is relatively low. The same configuration as the heat map image HM1 is also applied to the heat map image HM2 and the heat map image HM3.

In the all-findings integrated image MII, the first superimposition information of the nodule or the infiltrative shadow in which a specified color and a value of a specified opacity are applied to the pixel classified into the first class in the heat map image HM1 is superimposed on the medical image MI.

In addition, in the all-findings integrated image MII, the first superimposition information of the pneumothorax in which a specified color and a value of a specified opacity are applied to the pixel classified into the first class in the heat map image HM2 is superimposed on the medical image MI.

Further, in the all-findings integrated image MII, the first superimposition information of the pleural effusion in which a specified color and a value of a specified opacity are applied to the pixel classified into the first class in the heat map image HM3 is superimposed on the medical image MI.

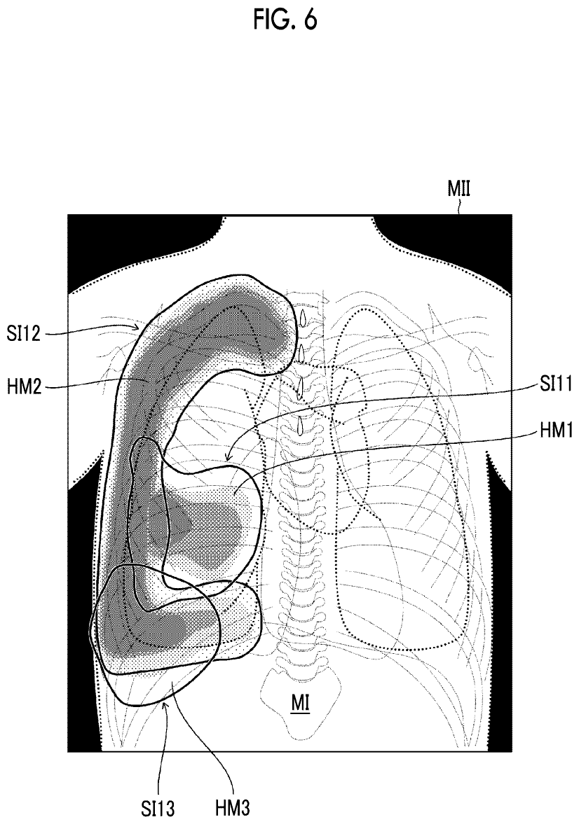

FIG. 6 is a schematic diagram of a medical image on which the first superimposition information is superimposed. FIG. 6 illustrates an enlarged view of the all-findings integrated image MII illustrated in FIG. 5. In FIG. 6, the heat map image HM1, the heat map image HM2, and the heat map image HM3 are schematically illustrated. In the heat map image HM1 and the like, a difference in degree of certainty is represented by applying a difference in hatching instead of a color.

First superimposition information SI11 of the nodule or the infiltrative shadow superimposed on the all-findings integrated image MII represents a boundary of a disease region of the nodule or the infiltrative shadow. First superimposition information SI12 of the pneumothorax superimposed on the all-findings integrated image MII represents a boundary of a disease region of the pneumothorax. First superimposition information SI13 of the pleural effusion superimposed on the all-findings integrated image MII represents a boundary of a disease region of the pleural effusion. That is, the first superimposition information SI11 of the nodule or the infiltrative shadow, the first superimposition information SI12 of the pneumothorax, and the first superimposition information SI13 of the pleural effusion represent a contour portion of an abnormal finding region.

A color that can be distinguished from the heat map image HM1 superimposed on the disease region of the nodule or the infiltrative shadow, the heat map image HM2 superimposed on the disease region of the pneumothorax, and the heat map image HM3 superimposed on the disease region of the pleural effusion is applied as the color applied to the first superimposition information SI11 of the nodule or the infiltrative shadow. The color applied to the first superimposition information SI12 of the pneumothorax and the color applied to the first superimposition information SI13 of the pleural effusion are also the same as the color applied to the first superimposition information SI11 of the nodule or the infiltrative shadow.

It is preferable that the color applied to the first superimposition information SI11 of the nodule or the infiltrative shadow, the color applied to the first superimposition information SI12 of the pneumothorax, and the color applied to the first superimposition information SI13 of the pleural effusion are colors that can be distinguished from each other.

As the value of the opacity applied to the first superimposition information SI11 of the nodule or the infiltrative shadow, a value of an opacity that can be distinguished from the heat map image HM1, the heat map image HM2, and the heat map image HM3 is applied.

The value of the opacity applied to the first superimposition information SI12 of the pneumothorax and the value of the opacity applied to the first superimposition information SI13 of the pleural effusion are also the same as the value of the opacity applied to the first superimposition information SI11 of the nodule or the infiltrative shadow.

It is preferable that the value of the opacity applied to the first superimposition information SI11 of the nodule or the infiltrative shadow, the value of the opacity applied to the first superimposition information SI12 of the pneumothorax, and the value of the opacity applied to the first superimposition information SI13 of the pleural effusion are values of the opacities that can be distinguished from each other.

The value of the opacity referred to here represents a degree of opacity of a specified color. The opacity is adjusted by changing a ratio of an area that transmits a color from a lower side to an area that does not transmit the color. In the present embodiment, a plurality of consecutive pixels representing the contour of the abnormal finding region are classified into the first class. A specified color is applied to the pixel classified into the first class, and further, a value of a specified opacity representing a ratio at which the specified color is not transmitted is applied. The value of the specified opacity is achieved by using a ratio of pixels that do not transmit the specified color to pixels that transmit the specified color for a plurality of pixels.

The specified color applied to each of the first superimposition information SI11, the first superimposition information SI12, and the first superimposition information SI13 described in the embodiment is an example of a first color, and the specified opacity is an example of a first opacity.

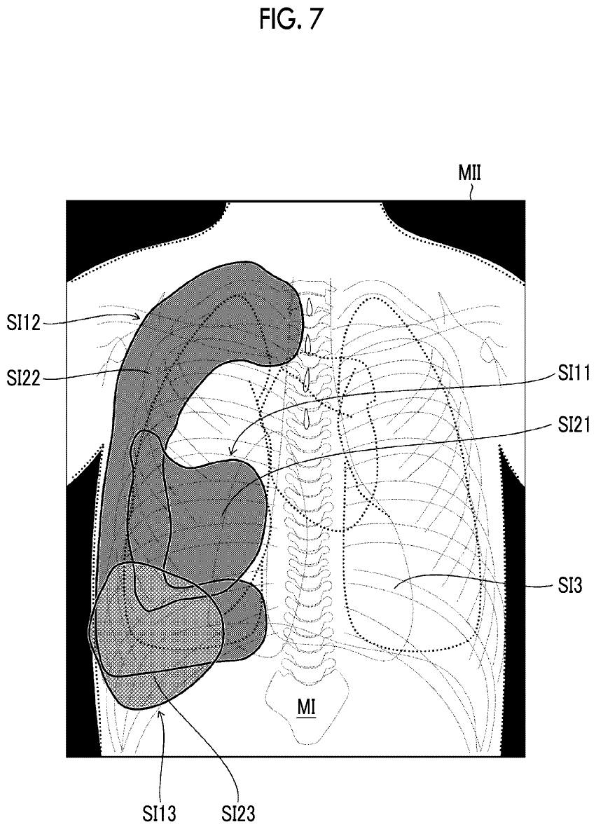

FIG. 7 is a schematic diagram of a medical image on which the second superimposition information and the third superimposition information are superimposed. A specified color and a value of a specified opacity value corresponding to the maximum value of the degree of certainty are applied to the pixel classified into the second class among the pixels constituting the medical image MI, and the specified color and the value of the specified opacity corresponding to the maximum value of the degree of certainty are superimposed on the medical image MI.

In the all-findings integrated image MII, second superimposition information SI21 of the nodule or the infiltrative shadow, second superimposition information SI22 of the pneumothorax, and second superimposition information SI23 of the pleural effusion are superimposed on the medical image MI on which the first superimposition information SI11 of the nodule or the infiltrative shadow, the first superimposition information SI12 of the pneumothorax, and the first superimposition information SI13 of the pleural effusion are superimposed.

Each of the second superimposition information SI21 of the nodule or the infiltrative shadow, the second superimposition information SI22 of the pneumothorax, and the second superimposition information SI23 of the pleural effusion has a specified color corresponding to the maximum value of the degree of certainty in the pixel classified into the second class and a specified opacity corresponding to the maximum value of the degree of certainty.

The color applied to the maximum value of the degree of certainty in the heat map image HM1 illustrated in FIGS. 5 and 6 is applied to the second superimposition information SI21 of the nodule or the infiltrative shadow illustrated in FIG. 7. The color applied to the maximum value of the degree of certainty in the heat map image HM2 is applied to the second superimposition information SI22 of the pneumothorax. The color applied to the maximum value of the degree of certainty in the heat map image HM3 is applied to the second superimposition information SI23 of the pleural effusion.

It is preferable that the value of the opacity applied to the second superimposition information SI21 of the nodule or the infiltrative shadow is less than the value of the opacity applied to the first superimposition information SI11 of the nodule or the infiltrative shadow. The value of the opacity applied to the second superimposition information SI21 of the nodule or the infiltrative shadow may be less than the value of the opacity applied to the first superimposition information SI12 of the pneumothorax, and may be less than the value of the opacity applied to the first superimposition information SI13 of the pleural effusion.

It is preferable that the value of the opacity applied to the second superimposition information SI22 of the pneumothorax is less than the value of the opacity applied to the first superimposition information SI2 of the pneumothorax. The value of the opacity applied to the second superimposition information SI22 of the pneumothorax may be less than the value of the opacity applied to the first superimposition information SI11 of the nodule or the infiltrative shadow, and may be less than the value of the opacity applied to the first superimposition information SI13 of the pleural effusion.

It is preferable that the value of the opacity applied to the second superimposition information SI23 of the pleural effusion is less than the value of the opacity applied to the first superimposition information SI13 of the pleural effusion. The value of the opacity applied to the second superimposition information SI23 of the pleural effusion may be less than the value of the opacity applied to the first superimposition information SI11 of the nodule or the infiltrative shadow, and may be less than the value of the opacity applied to the first superimposition information SI12 of the pneumothorax.

The specified color applied to each of the second superimposition information SI21, the second superimposition information SI22, and the second superimposition information SI23 described in the embodiment is an example of a second color, and the value of the opacity is an example of a second opacity.

FIG. 7 illustrates an all-findings integrated image MII with reference numeral SI3 representing the third superimposition information, in which third superimposition information SI3 is not generated and the input medical image MI is displayed as it is for the pixel classified into the third class. That is, in a case in which the medical image MI illustrated in FIG. 7 is displayed, the pixel of the medical image is displayed as it is for the pixel classified into the third class.

In a case in which a disease classified into the first class and a disease classified into the second class are present in one pixel, the first superimposition information for the disease classified into the first class is generated. In addition, in a case in which a disease classified into the first class and a disease classified into the third class are present in one pixel, the first superimposition information for the disease classified into the first class is generated. Further, in a case in which a disease classified into the second class and a disease classified into the third class are present in one pixel, the first superimposition information for the disease classified into the second class is generated.

### Effects of Medical Information Processing Apparatus and Medical Information Processing Method According to First Embodiment

The medical information processing apparatus 20 and the medical information processing method according to the first embodiment can obtain the following effects.

[1] In the medical image MI, a plurality of disease regions are detected, a degree of certainty of each disease is calculated for each pixel, and a heat map image representing a distribution of the degree of certainty is generated for each disease. A pixel of which a value of the degree of certainty is the same as a user threshold value for each disease and for each pixel is classified into a first class. First superimposition information to which a value of a first color and a value of a first opacity are applied is superimposed on the pixel classified into the first class. As a result, the first superimposition information indicating a contour of the disease region for each disease is superimposed on the medical image, and the visualization in which each of the plurality of disease regions is distinguished is achieved.

[2] As the color and the value of the opacity applied to the first superimposition information, a color and a value of an opacity with which the first superimposition information can be distinguished from the heat map image are applied. As a result, even in a case in which the plurality of disease regions overlap each other, the contour of each disease is distinguished from the medical image and the heat map.

[3] A pixel of which the degree of certainty exceeds the user threshold value is classified into a second class. For the pixel classified into the second class, second superimposition information to which a color and a value of an opacity corresponding to the maximum value of the degree of certainty in a plurality of pixels classified into the second class are applied is generated. The second superimposition information is superimposed on the medical image. As a result, the visualization of the disease region for each disease is achieved.

[4] A color of the maximum value of the degree of certainty in the heat map is applied as the color applied to the second superimposition information. As a result, the visualization of the disease region associated with the heat map for each disease is achieved.

[5] A value less than the opacity applied to the first superimposition information for each disease is applied as the value of the opacity applied to the second superimposition information. As a result, the first superimposition information is preferentially visualized.

[6] A pixel of which the degree of certainty is less than the user threshold value is classified into a third class. For the pixel classified into the third class, the medical image is displayed as it is. As a result, the display of the medical image in which the disease region and a non-disease region are distinguished from each other is implemented.

[7] The user threshold value acquisition unit acquires a user threshold value range including a value in the vicinity of the user threshold value. The classification unit classifies a pixel of which the degree of certainty of each disease for each pixel is within the user threshold value range into the first class. As a result, the number of pixels constituting the contour of the disease region is relatively increased, and the contour of the disease region is thickened and emphasized.

### First Modification Example of Visualization of Heat Map Image

In setting the value of the opacity for the pixel classified into the second class, in a case in which the pixel in the vicinity of the pixel classified into the second class is classified into the first class for any of all the diseases, a value of an opacity lower than the value of the opacity applied to the pixel of the first class may be applied.

As a result, the transmittance of the pixel of the first class that is visually recognized as the contour is relatively decreased, and the contour is emphasized. The value of the opacity lower than the value of the opacity applied to the pixel of the first class described in the embodiment is an example of a third opacity lower than the first opacity set for the pixel of the second class.

The pixel in the vicinity of the pixel classified into the second class may include a pixel adjacent to the pixel classified into the second class. The pixel in the vicinity of the pixel classified into the second class may be a pixel in a range of 2 pixels or more and 10 pixels or less from the pixel classified into the second class. An upper limit value of 10 pixels in the range of the pixels in the vicinity is merely an example and can be appropriately specified.

### Second Modification Example of Visualization of Heat Map Image

For each pixel constituting the heat map image illustrated in FIG. 6 and the like, the maximum value of the degree of certainty in a local region located within a specified range from each pixel is set as the value of the degree of certainty of each pixel, and the value of the degree of certainty of each pixel is updated. The range in the vicinity of each pixel may be from 10 pixels to 20 pixels in a case in which 1 pixel is 0.25 millimeters square.

In a case in which there is only one pixel having the maximum value of the degree of certainty, there is a concern that the disease region to which the pixel having the maximum value of the degree of certainty belongs is visually recognized as a normal region. Therefore, a dilation process is executed on the pixel having the maximum value of the degree of certainty, and the visibility of the region having the maximum value of the degree of certainty can be improved.

### Configuration Example of Medical Information Processing Apparatus According to Second Embodiment

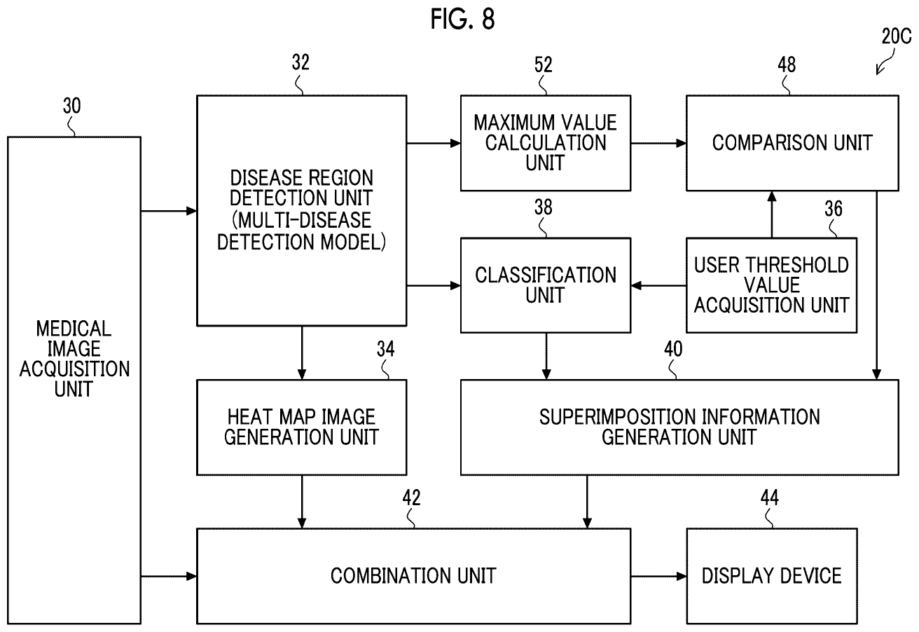

FIG. 8 is a functional block diagram showing an electric configuration of a medical information processing apparatus according to a second embodiment. A medical information processing apparatus 20C illustrated in FIG. 8 is configured by adding a maximum value calculation unit 52 and a comparison unit 48 to the medical information processing apparatus 20 illustrated in FIG. 2.

The maximum value calculation unit 52 calculates the maximum value of the degree of certainty of each disease. The comparison unit 48 compares the maximum value of the degree of certainty of each disease with the user threshold value and generates a comparison result indicating whether or not the maximum value of the degree of certainty of each disease exceeds the user threshold value.

In a case in which the comparison unit 48 generates a comparison result indicating that the maximum value of the degree of certainty of each disease exceeds the user threshold value, the superimposition information generation unit 40 generates text information representing a disease name and text information representing a maximum value of a degree of certainty associated with the disease name.

The medical information processing apparatus 20C may comprise a text information generation unit that generates text information representing a disease name and text information representing a maximum value of a degree of certainty associated with the disease name, separately from the superimposition information generation unit 40.

The combination unit 42 generates an all-findings integrated image MII2 in which text information representing a disease name and text information representing a maximum value of a degree of certainty associated with the disease name are superimposed on the medical image MI on which the heat map image HM1 or the like and the first superimposition information SI11 or the like are superimposed.

The display device 44 acquires a display signal representing an all-findings integrated image in which the heat map image HM1 or the like and the first superimposition information SI11 or the like are superimposed on the medical image MI and text information representing the maximum value of the degree of certainty of each disease is also superimposed thereon, and displays the all-findings integrated image on which the text information and the like are superimposed.

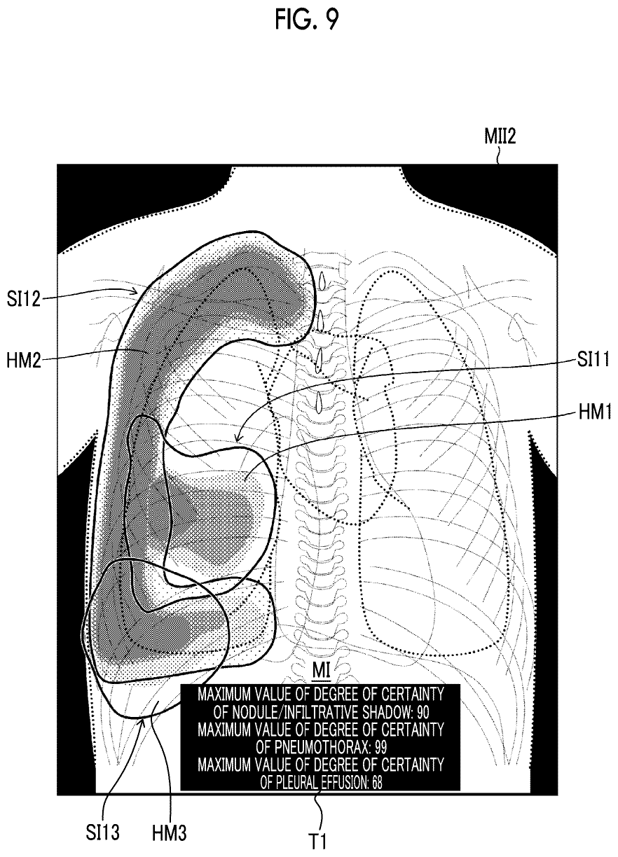

FIG. 9 is a schematic diagram of a medical image on which text information representing a maximum value of a degree of certainty of each disease is superimposed. In the all-findings integrated image MII2 illustrated in FIG. 9, text information T1 representing a maximum value of a degree of certainty of each disease is superimposed on the medical image MI. The text information T1 represents that a maximum value of a degree of certainty of a nodule or a pulmonary shadow is 90, a maximum value of a degree of certainty of a pneumothorax is 99, and a maximum value of a degree of certainty of a pleural effusion is 68.

The all-findings integrated image MII2 illustrated in FIG. 9 has the text information T1 superimposed on the medical image MI, but the all-findings integrated image MII2 may have a region where the text information T1 is disposed outside the medical image MI. The text information T1 can be disposed at any position in the all-findings integrated image MII2, but it is preferable that the text information T1 is disposed at a position that does not interfere with the visibility of the heat map image HM1 or the like and the first superimposition information SI11 or the like.

FIG. 9 illustrates the text information T1 in which text in white is superimposed on a background in black. Any color that can be distinguished from the background may be applied to the text of the text information T1. Any font and any size can be applied to the text of the text information T1.

The background of the text information T1 may be any color that can be distinguished from the text. The background of the text information T1 may be transparent or translucent.

A hardware configuration of an electric configuration of the medical information processing apparatus 20C according to the second embodiment is the same as the hardware configuration of the electric configuration of the medical information processing apparatus 20 illustrated in FIG. 3. In the medical information processing apparatus 20C, a maximum value calculation program applied to the maximum value calculation unit 52 and a comparison program applied to the comparison unit 48 are stored in the memory 112 illustrated in FIG. 3.

The processor 102 executes the maximum value calculation program to implement a maximum value calculation function. In addition, the processor 102 executes the comparison program to implement a comparison function.

In a medical information processing method applied to the medical information processing apparatus 20C according to the second embodiment, a maximum value calculation step executed by the maximum value calculation unit 52 and a comparison step executed by the comparison unit 48 are added to the flowchart illustrated in FIG. 4. In the composite image generation step S34, the text information T1 representing the disease name and the maximum value of the degree of certainty of each disease is generated. In the composite image display step S36, the composite image including the text information T1 is displayed.

### Effects of Medical Information Processing Apparatus According to Second Embodiment

According to the medical information processing apparatus 20C and the medical information processing method according to the second embodiment, in the all-findings integrated image MII2, the text information T1 representing the disease name and the maximum value of the degree of certainty of each disease is superimposed on the medical image MI. As a result, the visualization of the maximum value of the degree of certainty of each disease is achieved.

For example, in a case in which the medical image MI contains a plurality of disease regions for the same disease, the maximum degree of certainty among the plurality of disease regions for the same disease is visualized.

### Configuration Example of Medical Information Processing Apparatus According to Third Embodiment

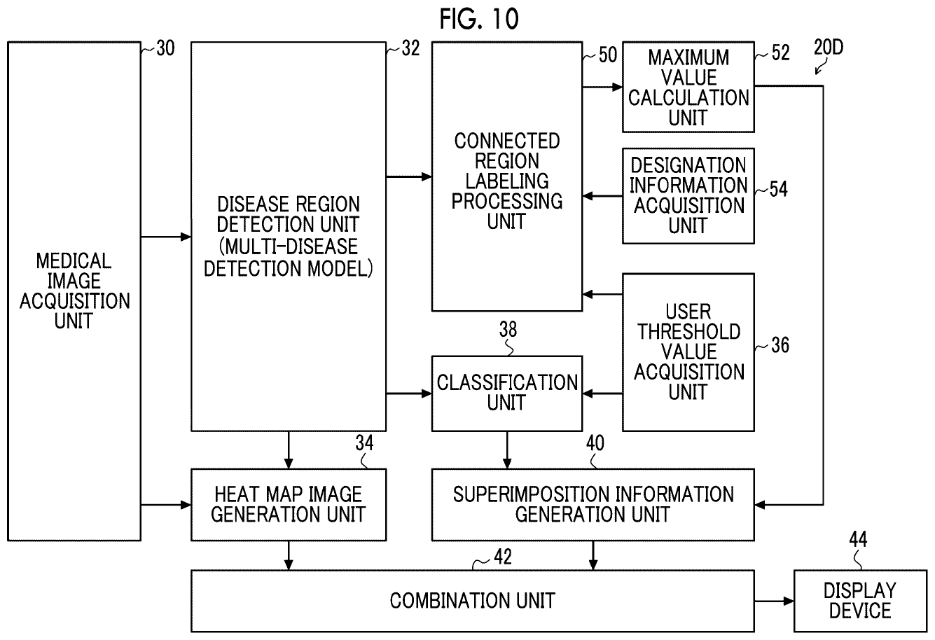

FIG. 10 is a functional block diagram showing an electric configuration of a medical information processing apparatus according to a third embodiment. A medical information processing apparatus 20D illustrated in FIG. 10 is configured by adding a connected region labeling processing unit 50, a maximum value calculation unit 52, and a designation information acquisition unit 54 to the medical information processing apparatus 20 illustrated in FIG. 2.

The connected region labeling processing unit 50 executes labeling processing on the disease region in which the degree of certainty of each disease exceeds the user threshold value. The connected region labeling processing unit 50 assigns a label for identifying the disease region to the disease region of each disease.

In a case in which a plurality of connected regions isolated from each other are present in a contour of a disease region composed of a plurality of pixels classified into the first class, the connected region labeling processing unit 50 assigns a different label to each of the connected regions.

In a case in which there is a connected region where disease regions of different diseases overlap each other, the connected region labeling processing unit 50 assigns a different label for each disease to the connected region where the disease regions of different diseases overlap each other.

The maximum value calculation unit 52 calculates the maximum value of the degree of certainty for each connected region to which the label is assigned. The maximum value calculation unit 52 stores the maximum value of the degree of certainty of each disease and the label of the region in association with each other for each connected region. In FIG. 10, a storage unit in which the maximum value of the degree of certainty of each disease is stored for each connected region is not illustrated.

The designation information acquisition unit 54 acquires position information of a pixel designated by the user operating a mouse. As the positional information of the pixel, coordinate values of a two-dimensional coordinate system defined for the medical image MI are applied. The designation information acquisition unit 54 transmits the acquired position information of the pixel to the connected region labeling processing unit 50.

The connected region labeling processing unit 50 specifies the connected region to which the pixel designated by the user operating the mouse belongs. The maximum value calculation unit 52 transmits the maximum value of the degree of certainty of each disease in the designated connected region to the superimposition information generation unit 40.

The superimposition information generation unit 40 generates text information representing the maximum value of the degree of certainty of each disease in the designated connected region. The medical information processing apparatus 20D may comprise a text information generation unit that generates the text information representing the maximum value of the degree of certainty of each disease in the designated connected region, separately from the superimposition information generation unit 40.

The combination unit 42 generates an all-findings integrated image in which the text information representing the maximum value of the degree of certainty of each disease in the connected region to which the pixel designated by the user operating the mouse is superimposed on the medical image MI on which the heat map image HM1 or the like and the first superimposition information SI11 or the like are superimposed.

The display device 44 acquires a display signal representing an all-findings integrated image in which the heat map image HM1 or the like and the first superimposition information SI11 or the like are superimposed on the medical image MI and text information representing the maximum value of the degree of certainty of each disease in the designated connected region is also superimposed thereon, and displays the all-findings integrated image on which the text information and the like are superimposed.

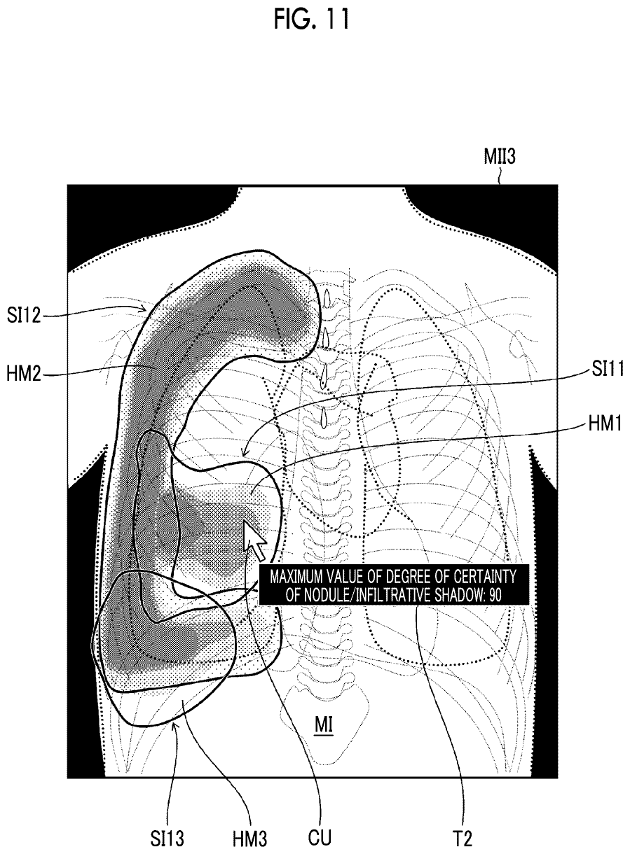

FIG. 11 is a schematic diagram of a medical image on which a maximum value of a degree of certainty of each disease in a designated region is superimposed. In an all-findings integrated image MII3 illustrated in FIG. 11, a cursor CU that moves in response to a user's mouse operation is displayed on the medical image MI.

In addition, in the all-findings integrated image MII3, text information T2 representing a maximum value of a degree of certainty of each disease in a connected region to which a pixel designated by using the cursor CU belongs is superimposed on the medical image MI.

FIG. 11 illustrates the all-findings integrated image MII3 in which a pixel in a disease region of a nodule or an infiltrative shadow is designated and the text information T2 representing that a maximum value of a degree of certainty of the nodule or the infiltrative shadow in a connected region to which the designated pixel belongs is 90 is superimposed on the medical image MI.

In a case in which the connected region is designated using the cursor CU, the connected region to which the pixel on which the cursor CU is superimposed belongs may be regarded as the designated connected region. The pixel on which the cursor CU is superimposed may be a pixel having the same coordinate value as a coordinate value of a tip position of the cursor CU. Any position of the cursor CU may be applied instead of the tip position of the cursor CU.

In the all-findings integrated image MII3 illustrated in FIG. 11, the text information T2 is superimposed within the range of the medical image MI. The all-findings integrated image MII3 may have a region where the text information T2 is disposed outside the medical image MI. The text information T2 can be disposed at any position in the all-findings integrated image MII3, but it is preferable that the text information T2 is disposed at a position that does not interfere with the visibility of the heat map image HM1 or the like and the first superimposition information SI11 or the like.

As with the text information Tl illustrated in FIG. 9, a background color, a text color, a font of the text, and a size of the text may be defined for the text information T2 shown in FIG. 11.

A hardware configuration of an electric configuration of the medical information processing apparatus 20D according to the third embodiment is the same as the hardware configuration of the electric configuration of the medical information processing apparatus 20 illustrated in FIG. 3. In the medical information processing apparatus 20D, a connected region labeling processing program applied to the connected region labeling processing unit 50, a maximum value calculation program applied to the maximum value calculation unit 52, and a designation information acquisition program applied to the designation information acquisition unit 54 are stored in the memory 112 illustrated in FIG. 3.

The processor 102 executes the connected region labeling processing program to implement a connected region labeling processing function. In addition, the processor 102 executes the maximum value calculation program to implement a maximum value calculation function. Further, the processor 102 executes the designation information acquisition program to implement a designation information acquisition function.

In a medical information processing method applied to the medical information processing apparatus 20D according to the third embodiment, a connected region labeling processing step executed by the connected region labeling processing unit 50, a maximum value calculation step executed by the maximum value calculation unit 52, and a designation information acquisition step executed by the designation information acquisition unit 54 are added to the flowchart illustrated in FIG. 4. In the composite image generation step S34, the text information T2 is generated that represents the maximum value of the degree of certainty of each disease in the connected region to which the pixel designated by the user operating the mouse belongs. In the composite image display step S36, the all-findings integrated image MII3 including the text information T2 is displayed.

### Effects of Third Embodiment

With the medical information processing apparatus 20D according to the third embodiment, the visualization of the disease name and the maximum value of the degree of certainty of each disease is achieved for the connected region to which the designated pixel designated by the user operating the mouse belongs.

### Configuration Example of Medical Information Processing Apparatus According to Fourth Embodiment

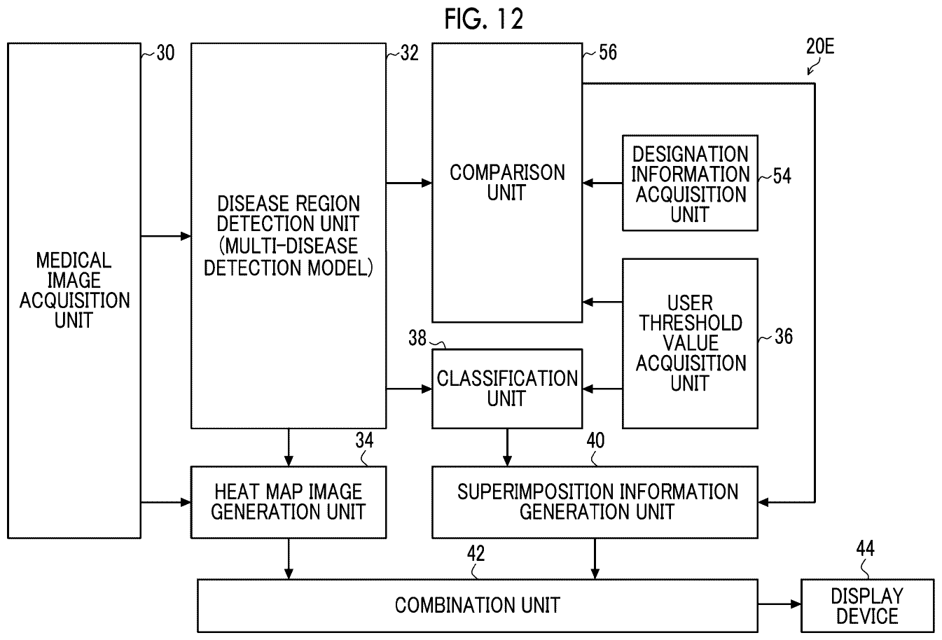

FIG. 12 is a functional block diagram showing an electric configuration of a medical information processing apparatus according to a fourth embodiment. A medical information processing apparatus 20E illustrated in FIG. 12 is configured by adding a designation information acquisition unit 54 and a comparison unit 56 to the medical information processing apparatus 20 illustrated in FIG. 2. The designation information acquisition unit 54 acquires information on a position in a medical image designated by the user operating the mouse.

The comparison unit 56 compares the degree of certainty of each disease with the user threshold value for the pixel designated by the user, and determines whether or not the degree of certainty of each disease exceeds the user threshold value. In a case in which the degree of certainty of each disease for the designated pixel exceeds the user threshold value, the comparison unit 56 transmits the degree of certainty of each disease in the designated pixel to the superimposition information generation unit 40.

The superimposition information generation unit 40 generates text information representing the degree of certainty of each disease in the pixel designated by the user. The medical information processing apparatus 20E may comprise a text information generation unit that generates the text information representing the degree of certainty of each disease in the pixel designated by the user, separately from the superimposition information generation unit 40.

The display device 44 acquires a display signal representing an all-findings integrated image in which the text information representing the degree of certainty of each disease in the pixel designated by the user is superimposed on the medical image, and displays the all-findings integrated image in which the text information representing the degree of certainty of each disease in the pixel designated by the user is superimposed.

In a case in which the pixel designated by the user has a degree of certainty exceeding the user threshold value for a plurality of diseases, the superimposition information generation unit 40 may generate text information representing the degree of certainty of each disease for each of the plurality of diseases.

An example of the text information representing the degree of certainty of each disease in the pixel designated by the user is the text information T2 illustrated in FIG. 11. The text information may be disposed at a position in contact with the cursor CU or may be disposed at a position away from the cursor CU.

A hardware configuration of an electric configuration of the medical information processing apparatus 20E according to the fourth embodiment is the same as the hardware configuration of the electric configuration of the medical information processing apparatus 20 illustrated in FIG. 3. In the medical information processing apparatus 20E, the designation information acquisition program and the comparison program are stored in the memory 112 illustrated in FIG. 3. The processor 102 executes the designation information acquisition program to implement a designation information acquisition function. The processor 102 executes the comparison program to implement a comparison function.

In a medical information processing method applied to the medical information processing apparatus 20E according to the fourth embodiment, a designation information acquisition step executed by the designation information acquisition unit 54 and a comparison step executed by the comparison unit 56 are added to the flowchart illustrated in FIG. 4. In the composite image generation step S34, the text information T2 representing the degree of certainty of each disease in the pixel designated by the user is generated. In the composite image display step S36, the all-findings integrated image MII3 including the text information T2 is displayed.

### Effects of Fourth Embodiment

With the medical information processing apparatus 20E according to the fourth embodiment, in a case in which the degree of certainty exceeds the user threshold value for the pixel designated by the user operating the mouse, the visualization of at least one of the disease name or the degree of certainty of each disease is achieved.

### Configuration Example of Medical Information Processing Apparatus According to Fifth Embodiment

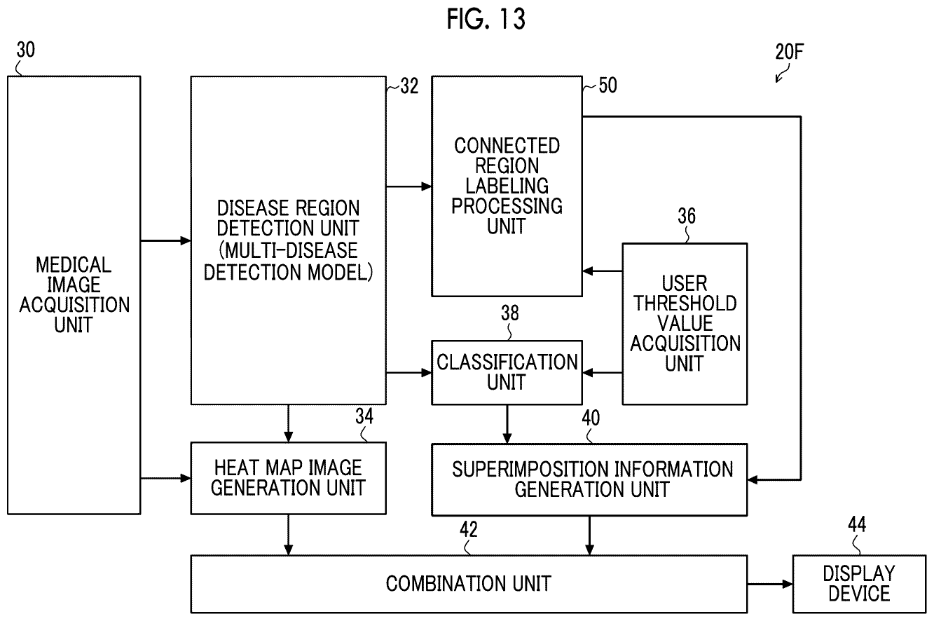

FIG. 13 is a functional block diagram showing an electric configuration of a medical information processing apparatus according to a fifth embodiment. The medical information processing apparatus 20F according to the fifth embodiment is applied in a case in which the user cannot operate the input device 16A such as the mouse.

The medical information processing apparatus 20F is configured by adding a connected region labeling processing unit 50 to the medical information processing apparatus 20 illustrated in FIG. 2. The medical information processing apparatus 20F uses the connected region labeling processing unit 50 to assign a label of each disease to each connected region where the value of the degree of certainty of each disease exceeds the user threshold value.

The connected region labeling processing unit 50 stores at least one of a disease name or a degree of certainty of each disease for each connected region in association with a label for each connected region. In FIG. 13, a memory in which at least any of the disease name or the degree of certainty of each disease is stored for each connected region is not illustrated.

The superimposition information generation unit 40 generates text information representing at least any of the disease name or the degree of certainty of each disease for each connected region. The combination unit 42 generates a composite image in which text information representing at least any of the disease name or the degree of certainty of each disease for each connected region is superimposed on the medical image MI. The combination unit 42 transmits a signal representing the generated composite image to the display device 44. The display device 44 displays the composite image.

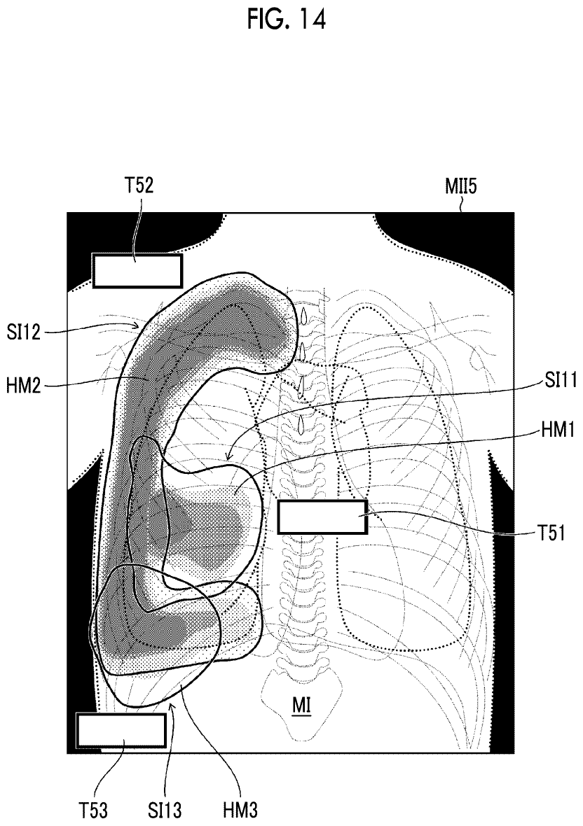

FIG. 14 is a schematic diagram of a medical image on which a disease name and a degree of certainty of each disease are superimposed for each connected region. In an all-findings integrated image MII5 illustrated in FIG. 14, text information T51, text information T52, and text information T53 are superimposed on the medical image MI.

The text information T51 includes text information representing at least one any a disease name or a degree of certainty in a connected region detected as a disease region of a nodule or an infiltrative shadow. The text information T52 includes text information representing at least any of a disease name or a degree of certainty in a connected region detected as a disease region of a pneumothorax. The text information T53 includes text information representing at least any of a disease name or a degree of certainty in a connected region detected as a disease region of a pleural effusion.

In FIG. 14, specific illustration of the disease name and the degree of certainty in each of the text information T51, the text information T52, and the text information T53 is omitted, and a background of the text information T51 and the like is illustrated.

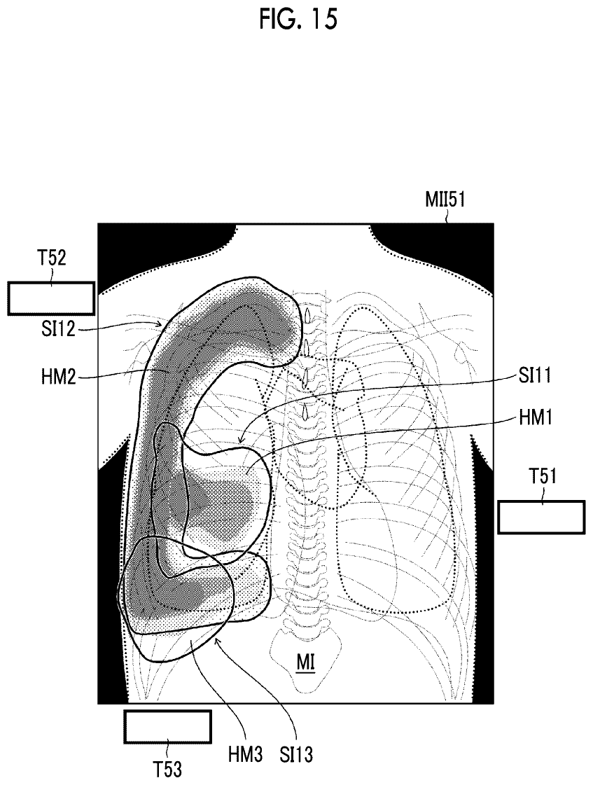

FIG. 15 is a schematic diagram showing a modification example of the text information illustrated in FIG. 14. In an all-findings integrated image MII51 illustrated in FIG. 15, the text information T51, the text information T52, and the text information T53 are disposed outside the region of the medical image MI. The text information T51, the text information T52, and the text information T53 may be disposed at a position superimposed on the medical image MI as long as the text information T51, the text information T52, and the text information T53 are outside the region of the examination target part in the medical image MI. For example, in a case in which the medical image MI is a chest X-ray image, the text information T51, the text information T52, and the text information T53 need only be disposed outside the region of the lung field. The disposition of the text information T51, the text information T52, and the text information T53 described in the embodiment is an example other than the detection target region in the medical image.

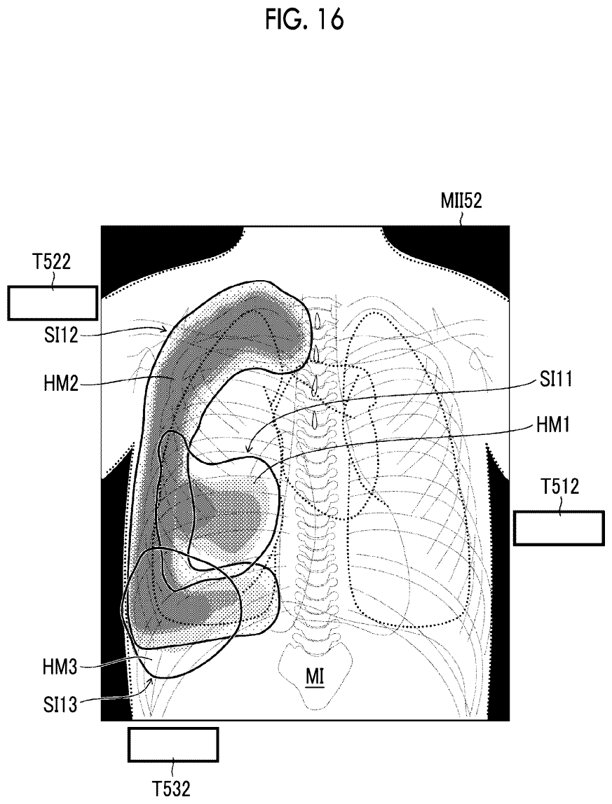

FIG. 16 is a schematic diagram showing another modification example of the text information illustrated in FIG. 14. In an all-findings integrated image MII52 illustrated in FIG. 16, text information T512, text information T522, and text information T532 are superimposed on the medical image MI.

The text information T512 includes all degrees of certainty of each disease in a connected region detected as a disease region of a nodule or an infiltrative shadow. The text information T522 includes all degrees of certainty of each disease in a connected region detected as a disease region of a pneumothorax. The text information T532 includes all degrees of certainty of each disease in a connected region detected as a disease region of a pleural effusion.

In FIG. 16, specific illustration of the degree of certainty and the like in each of the text information T512, the text information T522, and the text information T532 is omitted, and a background of the text information T512 and the like is illustrated.

A hardware configuration of an electric configuration of the medical information processing apparatus 20F according to the fifth embodiment is the same as the hardware configuration of the electric configuration of the medical information processing apparatus 20 illustrated in FIG. 3. In the medical information processing apparatus 20F, a connected region labeling processing program applied to the connected region labeling processing unit 50 is stored in the memory 112 illustrated in FIG. 3. The processor 102 executes the connected region labeling processing program to implement a connected region labeling processing function.

In a medical information processing method applied to the medical information processing apparatus 20F according to the fifth embodiment, a connected region labeling processing step executed by the connected region labeling processing unit 50 is added to the flowchart illustrated in FIG. 4. In the composite image generation step S34, the text information T51 and the like illustrated in FIG. 14 are generated. In the composite image display step S36, the all-findings integrated image MII5 including the text information T51 and the like is displayed.

### Effects of Fifth Embodiment

With the medical information processing apparatus 20F according to the fifth embodiment, in a case in which the user cannot operate the mouse, the visualization of at least one of the disease name or the degree of certainty of each disease is achieved for the connected region where the degree of certainty exceeds the user threshold value.

### Configuration Example of Medical Information Processing Apparatus According to Sixth Embodiment

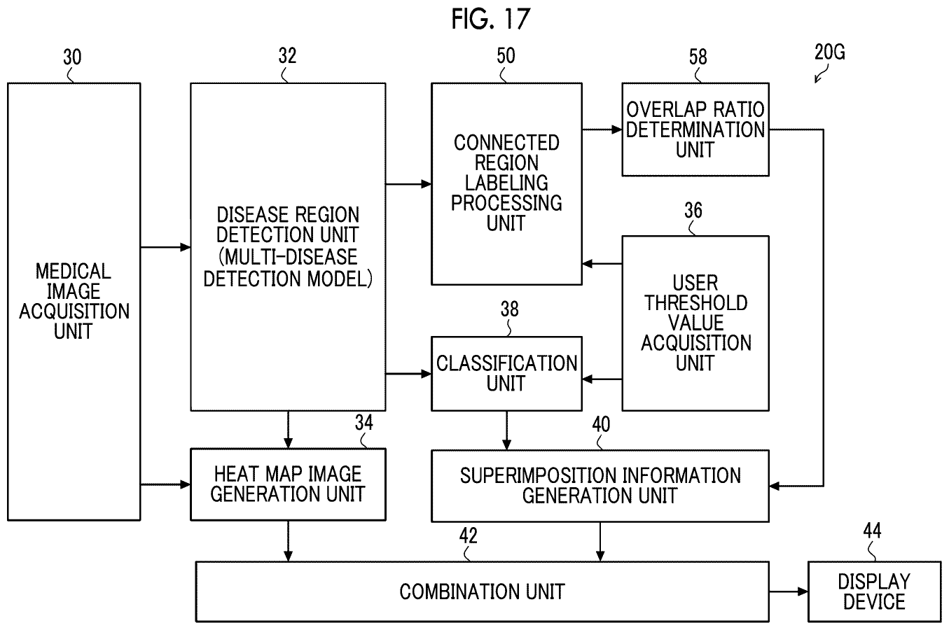

FIG. 17 is a functional block diagram showing an electric configuration of a medical information processing apparatus according to a sixth embodiment. In a case in which there is a portion where different disease regions overlap each other and an overlap ratio of each disease exceeds a specified value, a medical information processing apparatus 20G shown in FIG. 17 displays integrated text information in which disease names of the disease regions overlapping each other are integrated.

The medical information processing apparatus 20G is configured by adding a connected region labeling processing unit 50 and an overlap ratio determination unit 58 to the medical information processing apparatus 20 illustrated in FIG. 2. The connected region labeling processing unit 50 is the same processing unit as the connected region labeling processing unit 50 illustrated in FIG. 10, and assigns a label to a region where the degree of certainty of each disease exceeds the user threshold value.

In a case in which regions of different diseases overlap each other, the overlap ratio determination unit 58 determines whether or not the overlap ratio of each disease exceeds a specified value. The overlap ratio determination unit 58 transmits a signal representing the determination result to the superimposition information generation unit 40.

For the overlap ratio of each disease, a dice coefficient that takes a value in a range of 0 to 1.0 may be applied. The connected region labeling processing unit 50 may comprise an overlap ratio calculation unit that calculates the overlap ratio. The medical information processing apparatus 20G may comprise an overlap ratio calculation unit that calculates the overlap ratio, separately from the connected region labeling processing unit 50.

The specified value applied to the determination of the overlap ratio functions as a determination threshold value of the overlap ratio. As the specified value, any value greater than 0 and less than 1.0 may be applied. For example, any value of 0.5 or more and less than 1.0 may be applied as the specified value.

The specified value may be specified according to an area of the disease region. The specified value may be relatively increased in a case in which the area of the disease region is relatively large, and the specified value may be relatively decreased in a case in which the area of the disease region is relatively small.

In a case in which the overlap ratio of each disease exceeds a specified value, the superimposition information generation unit 40 generates integrated text information in which pieces of text information representing disease names of the diseases are integrated. The combination unit 42 generates an all-findings integrated image in which the integrated text information is superimposed on the medical image MI. The display device 44 displays the all-findings integrated image.

In a case in which the overlap ratio exceeds the specified value in all the plurality of diseases, the superimposition information generation unit 40 may generate the integrated text information. The superimposition information generation unit 40 may generate the integrated text information in a case in which the overlap ratio exceeds the specified value in at least one disease of the plurality of diseases.

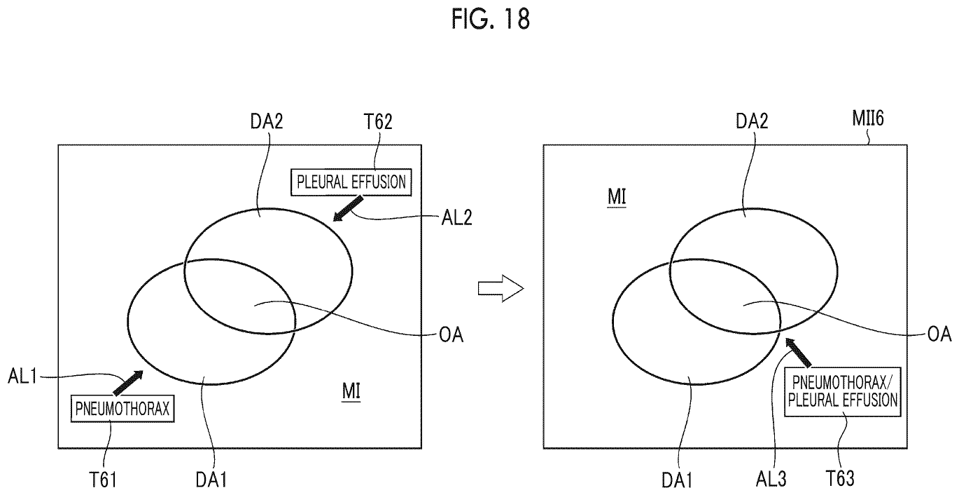

FIG. 18 is a schematic diagram of the integrated text information. In an all-findings integrated image MII6 illustrated in FIG. 18, a disease region DA1 of a pneumothorax and a disease region DA2 of a pleural effusion overlap each other, and integrated text information T63 generated instead of text information T61 and text information T62 representing each disease is superimposed on the medical image MI.

An arrow line AL1 from the text information T61 toward the disease region DA1 of the pneumothorax represents that the text information T61 corresponds to the disease region DA1 of the pneumothorax. In addition, an arrow line AL2 from the text information T62 toward the disease region DA2 of the pleural effusion represents that the text information T62 corresponds to the disease region DA2 of the pleural effusion.

In the integrated text information T63, the pneumothorax of the text information T61 and the pleural effusion of the text information T2 are written together. An arrow line AL3 output from the integrated text information T63 is directed to an overlapping region OA between the disease region DA1 of the pneumothorax and the disease region DA2 of the pleural effusion.

FIG. 18 illustrates a case in which two regions having different diseases, that is, the disease region DA1 and the disease region DA2 overlap each other, but, in a case in which three or more disease regions overlap each other, integrated text information in which pieces of the text information of the three disease regions are integrated may be generated.

A hardware configuration of an electric configuration of the medical information processing apparatus 20G according to the sixth embodiment is the same as the hardware configuration of the electric configuration of the medical information processing apparatus 20 illustrated in FIG. 3. In the medical information processing apparatus 20G, a connected region labeling processing program applied to the connected region labeling processing unit 50 and an overlap ratio determination program applied to the overlap ratio determination unit 58 are stored in the memory 112 illustrated in FIG. 3.

The processor 102 executes the connected region labeling processing program to implement a connected region labeling processing function. In addition, the processor 102 executes the overlap ratio determination program to implement an overlap ratio determination function.

In the medical information processing method applied to the medical information processing apparatus 20G according to the sixth embodiment, a connected region labeling processing step executed by the connected region labeling processing unit 50 and an overlap ratio determination step executed by the overlap ratio determination unit 58 are added to the flowchart illustrated in FIG. 4. In the composite image generation step S34, the integrated text information T63 corresponding to the plurality of disease regions is generated. In the composite image display step S36, the all-findings integrated image MII6 including the integrated text information T63 is displayed.

### Effects of Sixth Embodiment

With the medical information processing apparatus 20G according to the sixth embodiment, in a case in which a plurality of disease regions having different diseases overlap each other, the visibility of the disease name of each disease can be improved.

### Configuration Example of Medical Information Processing Apparatus According to Seventh Embodiment

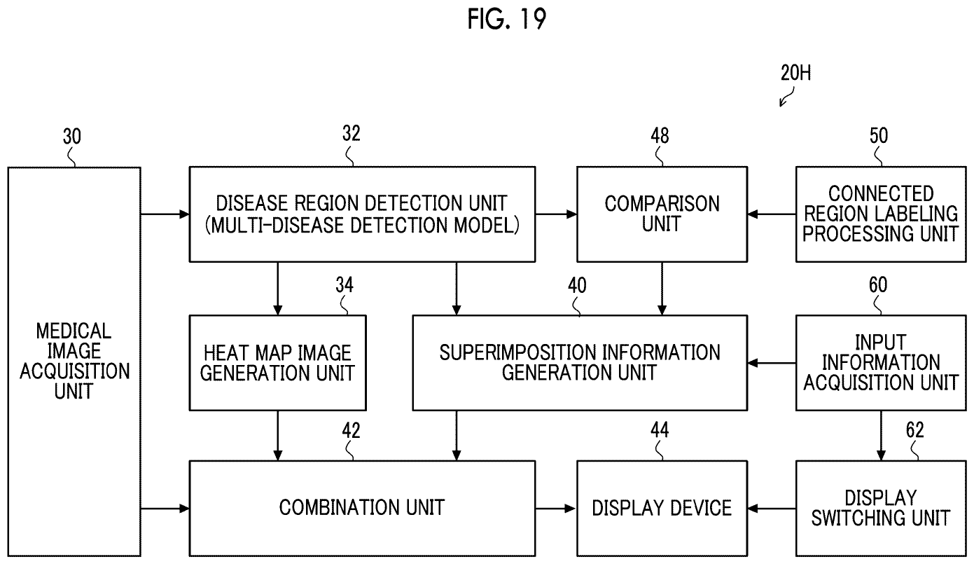

FIG. 19 is a functional block diagram showing an electric configuration of a medical information processing apparatus according to a seventh embodiment. A medical information processing apparatus 20H illustrated in FIG. 19 executes switching between the display of the visualization of the heat map image illustrated in FIG. 6 and the like and the display of the heat map image for each disease illustrated in FIG. 5 depending on input information from the user.

That is, the medical information processing apparatus 20H is configured by adding an input information acquisition unit 60 and a display switching unit 62 to the medical information processing apparatus 20 illustrated in FIG. 2. The input information acquisition unit 60 acquires a signal representing the input information input by the user using the input device 16A illustrated in FIG. 1. The display switching unit 62 switches the display of the display device 44 according to the input information acquired by using the input information acquisition unit 60.

A hardware configuration of an electric configuration of the medical information processing apparatus 20H according to the seventh embodiment is the same as the hardware configuration of the electric configuration of the medical information processing apparatus 20 illustrated in FIG. 3. In the medical information processing apparatus 20H, an input information acquisition program applied to the input information acquisition unit 60 and a display switching program applied to the display switching unit 62 are stored in the memory 112 illustrated in FIG. 3.

The processor 102 executes the input information acquisition program to implement an input information acquisition function. In addition, the processor 102 executes the display switching program to implement a display switching function.

In the medical information processing method applied to the medical information processing apparatus 20H according to the seventh embodiment, an input information acquisition step executed by the input information acquisition unit 60 and a display switching step executed by the display switching unit 62 are added to the flowchart illustrated in FIG. 4.

### Effects of Seventh Embodiment

With the medical information processing apparatus 20H according to the seventh embodiment, the user can selectively switch between the display of the visualization of the heat map image and the display of the heat map image for each disease.

### Function of Supporting Creation of Multi-Findings Report

The medical information processing apparatus 20 and the like may have a function of supporting creation of a multi-findings report. The multi-findings report is a report in which a plurality of findings included in one medical image are described. An example of the support for the creation of the multi-findings report is an example in which the text information generated by using the medical information processing apparatus 20 is automatically input to the multi-findings report.

The medical information processing apparatus 20 and the like comprise an anatomical structure information acquisition unit that acquires anatomical structure information from the medical image MI, a degree-of-certainty maximum value acquisition unit that acquires a maximum value of a degree of certainty of each disease, for a region for each anatomical structure, and a determination unit that determines whether or not the maximum value of the degree of certainty of each disease in the region for each anatomical structure exceeds a specified threshold value. The disease region detection unit 32 illustrated in FIG. 2 may be applied as the degree-of-certainty maximum value acquisition unit and the determination unit.

The medical information processing apparatus 20 and the like comprise a text information generation unit that generates text information representing a combination of an anatomical structure name, a disease name, and the maximum value of the degree of certainty in a case in which the maximum value of the degree of certainty of each disease in the region for each anatomical structure exceeds the specified threshold value. The generated text information is stored to be freely searchable using the anatomical structure name, the disease name, and the like as search keys.

The medical information processing apparatus 20 and the like comprise a text information transmission unit that transmits the text information representing the combination of the anatomical structure name, the disease name, and the maximum value of the degree of certainty to the user terminal device 16 in response to a request transmitted from the user terminal device 16 illustrated in FIG. 1. The superimposition information generation unit 40 may be applied as the text information generation unit and the text information transmission unit.

### Other Examples of Disease as Detection Target

In the disease region detection unit 32 illustrated in FIG. 2 and the like, a nodule, an infiltrative shadow, a pneumothorax, and a pleural effusion are detected as diseases, but the disease region detection unit 32 may detect a tumor, pulmonary tuberculosis, and the like as diseases.

The user terminal device 16 may have the functions of the medical information processing apparatus 20 illustrated in FIG. 1. That is, the user terminal device 16 may be provided with various processing units of the medical information processing apparatus 20 illustrated in FIG. 2 and the like. The user terminal device 16 comprises the memory 112 in which various programs illustrated in FIG. 3 are stored, and a processor may execute various programs to implement various functions.

The technical scope of the present invention is not limited to the scope described in the above embodiments. The configurations and the like in each embodiment can be appropriately combined between the respective embodiments without departing from the spirit of the present invention.

### EXPLANATION OF REFERENCES

10: medical information processing system
12: medical image capturing apparatus
14: medical image database
16: user terminal device
16A: input device
16B: display device
18: interpretation report database
20: medical information processing apparatus
20C: medical information processing apparatus
20D: medical information processing apparatus
20E: medical information processing apparatus
20F: medical information processing apparatus
20G: medical information processing apparatus
20H: medical information processing apparatus
22: network
30: medical image acquisition unit
32: disease region detection unit
34: heat map image generation unit
36: user threshold value acquisition unit
38: classification unit
40: superimposition information generation unit
42: combination unit
44: display device
48: comparison unit
50: connected region labeling processing unit
52: maximum value calculation unit
54: designation information acquisition unit
56: comparison unit
58: overlap ratio determination unit
60: input information acquisition unit
62: display switching unit
102: processor
104: computer-readable medium
106: communication interface
108: input/output interface
110: bus
112: memory
114: storage
120: medical image acquisition program
122: disease region detection program
124: heat map image generation program
126: user threshold value acquisition program
128: classification program
130: superimposition information generation program
132: combination program
134: degree-of-certainty acquisition program
AL1: arrow line
AL2: arrow line
AL3: arrow line
CI1: composite image
CI2: composite image
CI3: composite image
CU: cursor
DA1: disease region
DA2: disease region
HM1: heat map image
HM2: heat map image
HM3: heat map image
MI: medical image
MII: all-findings integrated image
MII2: all-findings integrated image
MII3: all-findings integrated image
MII5: all-findings integrated image
MII6: all-findings integrated image
MII51: all-findings integrated image
MII52: all-findings integrated image
OA: overlapping region
SI11: first superimposition information
SI12: first superimposition information
SI13: first superimposition information
SI21: second superimposition information
SI22: second superimposition information
SI23: second superimposition information
T1: text information
T2: text information
T51: text information
T52: text information
T53: text information
T61: text information
T62: text information
T63: text information
T512: text information
T522: text information
T532: text information
S10 to S36: each step of medical information processing method

What is claimed is:

1. An information processing apparatus comprising:
one or more processors; and
one or more memories that store instructions to be executed by the one or more processors,
wherein the one or more processors
acquire a medical image,
use a multi-disease detection model for detecting a plurality of disease regions from the medical image to acquire, for each disease, a degree of certainty representing at least any of presence or absence of a disease or a degree of the disease for each pixel of the medical image,
create degree-of-certainty distribution information for visualizing a distribution of the degree of certainty for each detected disease,
acquire a threshold value to be applied to the degree of certainty, and
perform control of superimposing at least any of a first color or a first opacity on the medical image for a pixel of a first image having the degree of certainty of the same value as the threshold value and displaying the superimposed image on a display device, for any disease.

2. The information processing apparatus according to claim 1,
wherein the one or more processors
acquire a threshold value range having a pre-specified upper limit value exceeding the threshold value and a pre-specified lower limit value less than the threshold value, and
classify a pixel having the degree of certainty within the threshold value range as the pixel of the first image.

3. The information processing apparatus according to claim 1,
wherein the one or more processors classify a pixel located at a distance within a specified range from a pixel classified as the pixel of the first image, as the pixel of the first image.

4. The information processing apparatus according to claim 1,
wherein the one or more processors perform control of, for a pixel of a second image of which the degree of certainty exceeds the degree of certainty in the pixel of the first image, superimposing and displaying at least any of a second color corresponding to a maximum value of the degree of certainty or a second opacity corresponding to the maximum value of the degree of certainty on the medical image, for any disease.

5. The information processing apparatus according to claim 4,
wherein the one or more processors set a third opacity lower than the first opacity for the pixel of the second image in a case in which a pixel adjacent to the pixel of the second image is classified as the pixel of the first image.

6. The information processing apparatus according to claim 1,
wherein the one or more processors perform control of causing the medical image to be displayed with a non-superimposed color and a non-superimposed opacity for a pixel of a third image of which the degree of certainty is less than the degree of certainty in the pixel of the first image, for any disease.

7. The information processing apparatus according to claim 1,
wherein the one or more processors
acquire a maximum value of the degree of certainty of each disease,
determine whether or not the maximum value of the degree of certainty exceeds the threshold value for each disease, and
perform control of displaying text representing a disease name and the maximum value of the degree of certainty in a case in which the maximum value of the degree of certainty exceeds the threshold value.

8. The information processing apparatus according to claim 1,
wherein the one or more processors
execute labeling processing on one or more connected regions including a plurality of consecutive pixels of which a value of the degree of certainty of each disease exceeds the threshold value,
determine whether or not a maximum value of the degree of certainty exceeds the threshold value for each connected region of each disease,
receive an input of a user who designates the connected region, and
perform control of displaying the maximum value of the degree of certainty of each disease in the designated connected region.

9. The information processing apparatus according to claim 1,
wherein the one or more processors
receive an input of a user who designates a pixel in the medical image, and
perform control of, in a case in which the degree of certainty of each disease in the designated pixel exceeds the threshold value, displaying a value of the degree of certainty.

10. The information processing apparatus according to claim 1,
wherein the one or more processors
execute labeling processing on one or more connected regions including a plurality of consecutive pixels of which a value of the degree of certainty of each disease exceeds the threshold value, and
perform control of displaying at least any of a disease name or the degree of certainty of the connected region for each connected region of each disease.

11. The information processing apparatus according to claim 10,
wherein the one or more processors perform control of displaying at least any of the disease name for each connected region or the degree of certainty of the connected region outside a detection target region in the medical image.

12. The information processing apparatus according to claim 1,
wherein the one or more processors
execute labeling processing on one or more connected regions including a plurality of consecutive pixels of which the degree of certainty of each disease exceeds the threshold value, and
perform control of displaying all the degrees of certainty of each disease for each connected region.

13. The information processing apparatus according to claim 1,
wherein the one or more processors
execute labeling processing on one or more connected regions including a plurality of consecutive pixels of which the degree of certainty of each disease exceeds the threshold value, and
perform control of displaying text information obtained by integrating a plurality of disease names in an overlapping portion of each disease in a case in which the overlapping portion exceeds a specified value, for each connected region of each disease.

14. The information processing apparatus according to claim 1,
wherein the one or more processors replace a value of a degree of certainty of a pixel constituting the degree-of-certainty distribution information with a maximum value of a degree of certainty in a local region located within a specified range from the pre-specified pixel.

15. The information processing apparatus according to claim 1,
wherein the one or more processors receive an input of a user for making a change to control of displaying the degree-of-certainty distribution information.

16. An operation method of an information processing apparatus, the method being executed by a computer functioning as the information processing apparatus and comprising:
a step of acquiring a medical image;
a step of using a multi-disease detection model for detecting a plurality of disease regions from the medical image to acquire, for each disease, a degree of certainty representing at least any of presence or absence of a disease or a degree of the disease for each pixel of the medical image;
a step of creating degree-of-certainty distribution information for visualizing a distribution of the degree of certainty for each detected disease;
a step of acquiring a threshold value to be applied to the degree of certainty; and
a step of performing control of superimposing at least any of a first color or a first opacity on the medical image for a pixel of a first image having the degree of certainty of the same value as the threshold value and displaying the superimposed image on a display device, for any disease.

17. A non-transitory, computer-readable tangible recording medium which records thereon a program for causing, when read by a computer functioning as an information processing apparatus, the computer to implement:
a function of acquiring a medical image;
a function of using a multi-disease detection model for detecting a plurality of disease regions from the medical image to acquire, for each disease, a degree of certainty representing at least any of presence or absence of a disease or a degree of the disease for each pixel of the medical image;
a function of creating degree-of-certainty distribution information for visualizing a distribution of the degree of certainty for each detected disease;
a function of acquiring a threshold value to be applied to the degree of certainty; and
a function of performing control of superimposing at least any of a first color or a first opacity on the medical image for a pixel of a first image having the degree of certainty of the same value as the threshold value and displaying the superimposed image on a display device, for any disease.

18. An information processing apparatus comprising:
one or more processors; and
one or more memories that store instructions to be executed by the one or more processors,
wherein the one or more processors
acquire a medical image,
use a multi-disease detection model for detecting a plurality of disease regions from the medical image to acquire, for each disease, a degree of certainty representing at least any of presence or absence of a disease or a degree of the disease for each pixel of the medical image,
acquire a threshold value to be applied to the degree of certainty,
acquire information on a region for each anatomical structure from the medical image,
acquire a maximum value of the degree of certainty of each disease, for the region for each anatomical structure, and
store a combination of an anatomical structure name, a disease name, and the maximum value of the degree of certainty in a case in which the maximum value of the degree of certainty of each disease in the region for each anatomical structure exceeds the threshold value.
